(19) United States
(12) Patent Application Publication
(10) Pub. No.: US 2024/0287627 A1
(43) Pub. Date: Aug. 29, 2024

(54) TEMPORALLY MULTIPLEXED IMAGING

(71) Applicant: MASSACHUSETTS INSTITUTE OF TECHNOLOGY, Cambridge, MA (US)

(72) Inventors: Yong Qian, Cambridge, MA (US); Edward S. Boyden, Cambridge, MA (US)

(73) Assignee: MASSACHUSETTS INSTITUTE OF TECHNOLOGY, Cambridge, MA (US)

(21) Appl. No.: 18/588,779

(22) Filed: Feb. 27, 2024

Related U.S. Application Data
(60) Provisional application No. 63/487,172, filed on Feb. 27, 2023.

(51) Int. Cl. C12Q 1/6897; A61B 5/00; C12N 15/85

(52) U.S. Cl. CPC C12Q 1/6897; C12N 15/85; A61B 5/0071

(57) ABSTRACT

The invention, in some aspects, include methods and systems for temporally multiplexed imaging (TMI).

### RELATED APPLICATIONS

This application claims benefit under 35 U.S.C. § 119(e) of U.S. Provisional application Ser. No. 63/487,172 filed Feb. 27, 2023, the disclosure of which is incorporated by reference herein in its entirety.

### GOVERNMENT INTEREST

This invention was made with government support under 1R01MH123977; R01DA029639; UF1NS107697; and 1R01MH114031 awarded by the National Institutes of Health. The government has certain rights in the invention.

### FIELD OF THE INVENTION

The invention, in some aspects, includes compositions and methods for multiplexed imaging of a plurality of fluorescent signals in living cells.

### BACKGROUND OF THE INVENTION

Fluorescence microscopy is important for measuring the temporal dynamics of cellular signals. An increasing emphasis is multiplexed fluorescence imaging—the ability to see many signals at once, in a living cell [(Linghu C, et al., Cell, (2020); 183(6):1682-98.e24), (Mehta S, et al., Nat Cell Biol, (2018); 20(10):1215-25)]). Without this ability, it is hard to determine the relationships between different signals, key to understanding how they interact to yield cellular computation, and how such computations go wrong in disease states. As a simple example, if when signal A is high in a given cell, signal B is low, and when signal A is low in a given cell, signal B is high, imaging of A and B in separate cells would miss out on this relationship. Traditionally, on conventional microscopes commonly available in biology, multiplexed fluorescent imaging relies on spectral differences between fluorophores associated with different signals, which limits the number of signals to a handful. Another recently developed multiplexing strategy called signaling reporter islands (SiRIs) enables potentially large numbers of signals to be imaged, by using self-assembling peptides to target dynamic fluorescent indicators to random, but stable, points throughout cells, so they can be imaged separately—a strategy called spatially multiplexed imaging (SMI) (Linghu C, et al., Cell, (2020); 183(6):1682-98.e24). However, SiRI technology relies on the availability of existing dynamic fluorescent indicators, raising the question of how gene expression monitoring, and other important biological experiments, could be implemented in a multiplexed way. Also, the use of space as a resource to facilitate imaging—a theme used in other recent imaging development such as expansion microscopy (Chen F, et al., Science, (2015); 347(6221):543-8)—raises the question that if time, and other nontraditional resources, could also be used to broaden the power of multiplexed imaging, ideally without requiring any exotic hardware to be obtained beyond those available to most biology groups.

### Reference to an Electronic Sequence Listing

The content of the electronic sequence listing (SequenceListing.xml; Size: 68,000 bytes; Date of Creation: Feb. 27, 2024) is herein incorporated by reference in its entirety.

### Summary of Certain Aspects of the Invention

According to an aspect of the invention, a method of measuring one or more cell activities is provided, the method including (a) expressing in a cell a plurality of reporter agents with independently selected temporal properties, wherein each of the expressed reporter agents is associated an independently selected gene; (b) obtaining simultaneous images of the plurality of expressed reporter agents; (c) linearly unmixing the obtained simultaneous images; and (d) analyzing the unmixed images, wherein the analysis includes measuring the one or more cell activities. In some embodiments, the reporter agents each includes an independently selected fluorophore. In some embodiments, the expressed reporter agent is capable of indicating expression of its associated independently selected gene. In some embodiments, the expressed reporter agent indicates expression of its associated independently selected gene In some embodiments, one or more of the plurality of the reporter agents includes an independently selected reversibly photoswitchable fluorescent protein (rsFP). In some embodiments, the reversibly photoswitchable fluorescent proteins (rsFPs) possess different off rates capable of indicating the expression of their independently selected genes. In some embodiments, the linearly unmixing of the images permits the plurality of reporter agent signals to be separated from each other. In some embodiments, plurality of reporter agent signals are separated from one or more of each other with the linearly unmixing of the images. In some embodiments, the linearly unmixing includes standard linear algebra. In some embodiments, the cell activity includes one or more of an enzyme activity in the cell, a cell cycle signal activity in the cell, or a second messenger activity in the cell. In some embodiments, the method also includes determining a relationship between two or more of the measured cell activities. In some embodiments, the determined relationship includes a relationship among two or more second messengers, kinases, and cell cycle signals in the cell. In some embodiments, the cell is a vertebrate cell. In some embodiments, the cell is a mammalian cell. In some embodiments, the cell is a human cell. In some embodiments, the cell is one or more of a neuron, a CNS cell, a PNS cell, a muscle cell, an endocrine cell, an immune system cell, an epidermal cell, a kidney cell, a liver cell, and a cardiac cell.

According to another aspect of the invention, a temporally multiplexed imaging (TMI) system is provided, the system including an encoded plurality of reporter agents with independently selected temporal properties expressed in a cell, wherein each of the expressed reporter agents is associated with an independently selected gene, and each expressed reporter agent indicates expression of its associated independently selected gene. In some embodiments, the reporter agents include independently selected fluorophores. In some embodiments, one or more of the plurality of the reporter agents each includes an independently selected reversibly photoswitchable fluorescent protein (rsFP). In some embodiments, the reversibly photoswitchable fluorescent proteins (rsFPs) possess different off rates capable of indicating the expression of their independently selected genes. In some embodiments, the reporter agents are expressed in a cell and images of the reporter agents are obtained. In some embodiments, the obtained images are linearly unmixed. In some embodiments, the linearly unmixing separates the plurality of reporter agent signals from each other. In some embodiments, the linearly unmixing includes standard linear algebra. In some embodiments, the linearly unmixed images are analyzed. In some embodiments, the analysis includes measurement of one or more of a cell activity in the cell. In some embodiments, the activity includes one or more of an enzyme activity in the cell, a cell cycle signal activity in the cell, or a second messenger activity in the cell. In some embodiments, the measurement of two or more of the cell activities determines a relationship between the two or more of activities in the cell. In some embodiments, the determined relationship includes a relationship among two or more second messengers, kinases, and cell cycle signals in the cell. In some embodiments, the cell is a vertebrate cell. In some embodiments, the cell is a mammalian cell. In some embodiments, the cell is a human cell. In some embodiments, the cell is one or more of a neuron, a CNS cell, a PNS cell, a muscle cell, an endocrine cell, an immune system cell, an epidermal cell, a kidney cell, a liver cell, and a cardiac cell.

### BRIEF DESCRIPTION OF THE DRAWINGS

FIG. 1A-C provides schematic diagrams illustrating principle of temporally multiplexed imaging (TMI). (FIG. 1A) Multiple reversibly photoswitchable fluorescent proteins (rsFPs, denoted 1 through 3), of the same color, are expressed in cells, with different amounts in each cell (and potentially in each part of a cell, FIG. 1A subsections a to d represent different combination of rsFP1 (top), rsFP2 (middle), and rsFP3 (bottom) in each of a-d in FIG. 1A. They are unable to be distinguished spectrally by conventional microscopes. (FIG. 1B) These rsFPs, however, behave differently over time due to differences in their easily measured temporal properties (i)—in this example, photoswitching during continuous imaging (e.g. different off-switching kinetics; the reference traces f₁ to f₃ are obtained individually, in a separate calibration experiment, under the same imaging conditions as used for the actual experiment). The fluorescence trace of each pixel is a linear combination of all the rsFP traces at that pixel, each scaled by the number of fluorophores of that kind present (ii). (FIG. 1C) The fluorescence intensity of each rsFP at each pixel is obtained via standard linear unmixing of the fluorescence trace, using the reference traces in FIG. 1B. The resultant images can then be pseudo-colored for easy visualization, by giving each rsFP a different digital color in the software.

FIG. 2A-F provides diagrams and graphs illustrating temporally multiplexed imaging of six green fluorescent proteins (FPs). (FIG. 2A) Green rsFPs can be switched to “off” states during imaging with continuous excitation at ˜480 nm and switched to “on” states by a pulse of purple light (˜400 nm). (FIG. 2B) Reference traces of rsFastGreen-E [trace (6)], rsEGFP2-E [trace (5), rsFastLime [trace (4)], Skylan-62A [trace (3)], Dronpa [trace (2)], and YFP [trace (1)], in U2OS cells (fixed cells, for this figure's validation experiment), n=10-15 cells from three movies from one culture. Data are shown as mean±standard deviation (SD). Illumination: 488 nm at 40 mW/mm². Due to their distinct switching kinetics, the six spectrally similar FPs could be unmixed via linear decomposition. (FIG. 2C) Co-expression of ER-targeted rsGreenF-E, nucleus targeted rsEGFP-E, mitochondria targeted rsFastLime, tubulin targeted Skylan-62A, actinin targeted dronpa, and lysosome targeted YFP in U2OS cells. A 70-frame movie of fixed U2OS cells was taken over 3.8 seconds and six images were obtained via linearly unmixing the fluorescence trace at each pixel in the movie using the reference traces in FIG. 2B. Scale bar, 20 μm. (FIG. 2D) Representative merged image of live NIH/3T3 cells with expression of 6 green FPs (not fused to targeting tags) from six images unmixed from the source movie. Each cell was transfected with exactly one FP (see Methods in Examples section for details). Scale bar, 50 μm. FIG. 2D shows at (1) rsGreenF-E; at (2) rsEGFP2-E; at (3) rsFastLime; at (4) Skylan62A; at (5) Dronpa and at (6) YFP. Fluorescence cross-talk between two FPs was calculated based on the unmixed images. (FIG. 2E) FP-FP crosstalk was calculated from the linear decomposition coefficients extracted from 15 images from two cultures experimented upon as in FIG. 2D, and expressed as a percentage of the true FP brightness for a given cell (recall each cell has exactly one FP, providing ground truth). Values are shown as mean±SD; color represents the mean. (FIG. 2F) Accuracy of linear decomposition of temporally multiplexed movies into individual FP images was simulated by taking cell images and populating the pixels with traces that are scaled versions of the curves of panel FIG. 2B, followed by unmixing and comparing to the ground truth—which, being simulated, are exactly known. See Methods for details. Each data point represents one cell. The X-axis value of each data point represents the ground-truth fluorescence of a FP divided by the total ground-truth fluorescence summed over all FPs. The Y-axis value of each data point represents the fluorescence difference between the unmixed image and its corresponding ground truth, divided by the ground truth.

FIG. 3A-G provides graphs and images illustrating temporally multiplexed imaging of four red FPs. (FIG. 3A) Red rsFPs switch “off” with excitation at ˜560 nm and switch “on” with irradiation of either blue (480 nm) or purple light (400 nm). (FIG. 3B) Reference traces of rsTagRFP (trace with “+ marks”), rsFusionRed1 (trace with no marker), rScarlet (trace with “o” marks), and mCherry (trace with “x” marks) in U2OS cells (fixed cells, for this figure's validation experiment). Traces were collected from 10-15 cells from one culture, and are shown as mean±SD. Illumination: 561 nm at 50 mW/mm². (FIG. 3C) Co-expression of nucleus targeted rsTagRFP, vimentin targeted rsFusionRed1, actinin targeted rScarlet, and mitochondria targeted mCherry in U2OS cells. A 70-frame movie of fixed U2OS cells was taken in 8.6 seconds and images of the four FPs were obtained via linearly unmixing the fluorescence trace at each pixel in that movie using the reference traces in FIG. 3B. Scale bar, 20 μm. (FIG. 3D) Representative merged image of live NIH/3T3 cells with expression of four red FPs (not fused to targeting tags) from four images unmixed from the source movie. Each cell was transfected with exactly one FP (see Methods for details). Scale bar, 50 μm. FIG. 3D shows at (1) MCherry; at (2) rScarlet; at (3) rsFusionRed1; and (4) rsTagRFP. Fluorescence cross-talk between two FPs were calculated based on the unmixed images. (FIG. 3E) FP-FP crosstalk was calculated from the linear decomposition coefficients extracted from six images from two cultures experimented upon as in FIG. 3D, and expressed as a percentage of the true FP brightness for a given cell (recall each cell has exactly one FP). Values are shown as mean±SD; color represents the mean. (FIG. 3F) Accuracy of linear decomposition of temporally multiplexed movies into individual FP images was simulated by taking cell images and populating the pixels with traces that are scaled versions of the curves of panel FIG. 3B, followed by unmixing and comparing to the ground truth—which, being simulated, are exactly known. See Methods in Examples section herein for details. Each data point represents one cell. The X-axis value of each data point represents the ground-truth fluorescence of a FP divided by the total ground-truth fluorescence. The Y-axis value of each data point represents the fluorescence difference between the unmixed image and its ground truth, divided by the ground truth. (FIG. 3G) Single-color “brainbow” in the larval zebrafish brain, based on temporally multiplexed imaging of rScarlet, rsTagRFP and rsFusionRed1. Middle, representative raw fluorescence image of zebrafish larvae hindbrain; right, brainbow-like image obtained via linear decomposition, showing the same area as the image in the middle panel. Scale bar, 20 μm.

FIG. 4A-H provides diagrams, graphs, and images illustrating temporally multiplexed imaging allows simultaneous observation of many signals at once, in diverse biological contexts. (FIG. 4A) Diagram of cell cycle indicators (known as fluorescent, ubiquitination-based cell cycle indicator 4, abbreviated FUCCI4). Dronpa, YFP, rsGreenF-E and Skylan62A were fused to cell cycle regulated proteins Cdt1₃₀₋₁₂₀, SLBP₁₈₋₁₂₆, Geminin₁₋₁₁₀, and histone H1.0 respectively. Analogous to the original FUCCI4, G1-S transition is reported by the emergence of rsGreenF-E fluorescence while YFP fluorescence persists, S-G2 transition is marked by the fluorescence loss of YFP and stable rsGreenF-E fluorescence. Chromosome condensation, labeled by Skylan62A, indicates the M phase; finally, loss of rsGreenF-E fluorescence and the appearance of Dronpa and YFP fluorescence means the beginning of the G1 phase. (FIG. 4B) Tracking of cell cycle phases of NIH/3T3 cells via time-lapse imaging using single-color FUCCI4. A mother cell dividing into two daughter cells was captured during an 11-hour imaging session. Scale bar, 20 μm. (FIG. 4C) Fluorescence traces of Dronpa-Cdt1₃₀₋₁₂₀ (open circles), rsGreenF-E-Geminin₁₋₁₁₀ (“x” marks on trace), and YFP-SLBP₁₈₋₁₂₆ (closed circles) of 3 cells over cell divisions, tracing one arbitrary daughter cell from each mother cell after M. Fluorescence was normalized to maximum value. Magenta bars indicate observation of chromosome condensation. Cell-cycle phases were assigned based on the principles in FIG. 4A. (FIG. 4D) Diagram of temporally multiplexed imaging-based kinase translocation reporters (KTRs). A KTR contains a kinase docking site, a phospho-inhibited bipartite nuclear localization signal (bNLS) containing phosphorylation sites (P sites), a phospho-enhanced nuclear export signal (NES) containing P sites, and an rsFP. When the corresponding kinase is inactive, the KTR protein is unphosphorylated and is nuclear enriched. When the corresponding kinase is active, the KTR protein is phosphorylated and excluded from the nucleus. (FIG. 4E) Four kinase sensors were made using four green FPs: an rsFastLime-based ERK sensor, an rsGreenF-E-based JNK sensor, a Dronpa-based P38 sensor, and a Clover-based PKA sensor. NIH/3T3 cells expressing all four KTRs and H2B-TagBFP as a nuclear marker were imaged and stimulated with mouse basic fibroblast growth factor 2 (bFGF2, 20 ng/ml), which is known to drive mitogen-activated proteins kinases (such as JNK, P38 and ERK) and PKA. Representative cell is shown (four pseudo-channels unmixed from green channel and one blue channel) at indicated time points. Scale bar, 20 μm. (FIG. 4F) Activity traces of the four kinases from the representative cell in FIG. 4E. R, cytoplasmic intensity to nuclear intensity ratio. The change in fluorescence of the sensors is plotted as ΔR/R₀. (FIG. 4G) Averaged traces of four kinase activities recorded from NIH/3T3 cells, with 20 ng/ml bFGF2 stimulation at t=0 min; n=16 cells from two culture batches. Data are shown as mean±standard error of the mean (SEM). Wilcoxon rank sum tests were run between the averaged values from t=−6 min to t=0 min and averaged values from t=12 min to t=18 min for PKA KTR and JNK KTR. Wilcoxon signed-rank tests were run between the averaged values from t=−6 min to t=0 min and averaged values from t=48 min to t=54 min for ERK KTR and P38 KTR. (FIG. 4H) Averaged traces of four kinase activities recorded from NIH/3T3 cells, with stimulation of 50 μM forskolin at t=0 min; n=10 cells from two culture batches. Data are shown as mean±SEM. Wilcoxon rank sum tests were run between the values from t=−6 min to t=0 min and t=30 min to t=36 min for all KTRs. Throughout the figure: *p<0.05; **p<0.01; ***p<0.001, ****p<0.0001.

FIG. 5A-F provides graphs and diagrams illustrating combined temporal and spectral multiplexing for simultaneous imaging of large numbers of signals within single cells. (FIG. 5A-C) Simultaneous observation of cell cycle phase changes and kinase activity. (FIG. 5A) Left, diagram of combined use of green rsFP-based FUCCI4 with TagBFP2-based CDK2 reporter and mCherry-based CDK4/6 reporter. Right, schematic of CDK activity in different cell cycle phases. The CDK activity progression during S and G2 phases was unknown (dashed line) previously. (FIG. 5B-C) Plots of CDK2 (FIG. 5B) and CDK4/6 (FIG. 5C) activity as a function of different stages (early, middle, and late stage) of all four cell cycle phases. CDK2 and CDK4/6 activities were calculated as follows. CDK2 activity was calculated as cytoplasm fluorescence to nucleus fluorescence ratio; CDK4/6 activity was calculated using the same way as CDK2 activity before processing as in the publication (Yang H W, et al., Elife [Internet], (2020); 9) without normalization. Each cell cycle phase was divided into early, middle, and late stages evenly and CDK activity of a given stage was obtained by averaging all CDK values in that stage (see Methods). Data are shown as connected mean±SD with all individual values plotted as dots (grey, dots of the same cell cycle phase of the same cell are connected); n=25 cells from five culture batches (not all cells exhibited all complete phases). Wilcoxon signed-rank test with Holm-Bonferroni correction, ****p <0.0001, ***p<0.001, **p<0.01, *p<0.05, n.s., not significant. (FIG. 5D) Superimposed plots of CDK2 and CDK4/6 activity. Data points were from the mean values of the plot in FIG. 5B-C, and then normalized between 0 and 1 for temporal comparisons. (FIG. 5E) Schematic image providing details of signals used for simultaneous imaging of seven signals within single NIH/3T3 cells. (FIG. 5F) Graphs showing results of combined use of green rsFP-based KTRs with BlueCKAR, Pink Flamindo and NIR-GECO2G. Signals of PKA, P38, ERK, JNK, Ca²⁺, cAMP, and PKC under stimulation of 50 μM forskolin (added at t=8 min), and 100 ng/ml PMA (added at t=44 min); n=7 cells from 2 culture batches, values are shown as mean±SEM. Signals from individual cells are shown in FIG. 13.

FIG. 6A-D provides graphs and schematic diagrams illustrating development of new green rsFPs for temporally multiplexed imaging. (FIG. 6A) Off-switching traces of a series of Skylan mutants obtained in HEK293FT cells, rsFastLime and Dronpa were also tested under the same conditions for reference. Averaged values are shown, n=8-12 cells from one culture. Excitation: 475/28 nm at 15 mW/mm². Skylan62A (indicated by triple stars) was the winner of the screening. (FIG. 6B) Crystal structure of mEos (PDB: 3S05). The chromophore of mEos is formed by the tripeptide HYG. Inspired by the engineering of photoswitchable Skylan-NS from mEos3.1, which was achieved by mutating His62 (highlighted by dashed circle) to Leu, a new photoswitchable FP, Skylan62A was developed from Skylan-NS by mutating Leu 62 to Ala. The off-switching rate of Skylan62A is in between that of Dronpa and rsFastLime, which makes Skylan62A a good candidate for temporally multiplexed imaging with rsFastLime and Dronpa. (FIG. 6C) Absorption (darkest trace), excitation (lighter trace), and emission (trace with open circles) spectra of Skylan62A. (FIG. 6D) Left, structure of rsGreenF-Enhancer (rsGreenF-E) and rsEGFP2-Enhancer (rsEGFP2-E). Right, off-switching traces of rsGreenF and rsEGFP2 with and without enhancer recorded in HEK293FT cells. rsFastLime was also tested under the same conditions for reference. Averaged values are shown, n=6-10 cells from one culture. Excitation: 475/28 nm at 5 mW/mm². The addition of a nanobody enhancer increases the photoswitching kinetics of rsGreenF and decreases the photoswitching rate of rsEGFP2. rsGreenF-E and rsEGFP2-E (indicated by triple stars) were then chosen for temporally multiplexed imaging.

FIG. 7A-B provides schematic diagram and graphs illustrating evaluation of TMI for subcellular labeling with six green FPs. (FIG. 7A) Experimental design for evaluating temporal multiplexing using green FPs. A FLAG tag was added to each of the six subcellularly targeted constructs. In the first experiment, as shown on the upper panel, each FLAG-tagged construct was expressed individually in U2OS cells. 48 hours post transfection, cells were fixed and immuno-stained with Alexa647 dye. Two snapshot images were taken from green channel (excitation: 488 nm, emission: 525/30 nm) and far-red channel (excitation: 637 nm, emission: 700/50 nm) followed by colocalization test and calculations of Pearson's correlation values. In the second experiment, as shown on the bottom panel, one FLAG-tagged construct was coexpressed with other five constructs without a FLAG tag in U2OS cells, 48 hours post transfection; a 70-frame movie was taken from the green channel and a snapshot image was taken from the far-red channel. After signal unmixing, six images were obtained from the green channel. The unmixed image of the FLAG-tagged construct was then co-localized with the image obtained from far-red channel followed by calculations of another set of Pearson's correlation values. The two sets of Pearson's correlation values were then compared for each FP. (FIG. 7B) Bar plots of Pearson's correlation values of each FP obtained in FIG. 7A. n=3 to 6 images from two culture batches. Data are shown as mean±SD with individual values plotted as dots. Unpaired t test was run between two sets of Pearson's correlation values for each FP; n.s., no significance.

FIG. 8A-B provides images illustrating simulation of temporally multiplexed imaging of six green FPs. A 70-frame time lapse image was generated using a pre-acquired fluorescence image of NIH/3T3 cells and the off-switching traces in FIG. 2B. The fluorescence components of the six FPs in each cell were randomly assigned. Different cells have different fluorescence combinations of the six FPs. Poisson noise was added to each pixel to mimic real images. (FIG. 8A) Simulated movie showing cells that express six green rsFPs. (FIG. 8B) After signal unmixing, six images were obtained for each FP. The images were then compared with ground-truth images. Pearson's correlation values between unmixed images and ground-truth images are shown. Detailed value comparisons are shown in FIG. 2F.

FIG. 9A-C provides schematic images and graphs illustrating development of a new red rsFP with a slow photo-switching rate for temporally multiplexed imaging. (FIG. 9A) Schematic illustration of the engineering of rScarlet from mScarlet. Left, crystal structure of mScarlet, the chromophore is shown in orange. Two rounds of evolution were performed before photoswitchable rScarlet was selected. rScarlet accumulates seven mutations compared to mScarlet, which is highlighted in the crystal structure on the right. The mutations from round one are highlighted with darker shade, the mutation from round two is highlighted with lighter shade. (FIG. 9B) Photoswitching traces of rScarlet in HEK293FT cells, n=3 cells from one culture. Data are shown as mean±SD. Excitation: 555/28 nm at 9.4 mW/mm², On-switching: 475/28 nm at 9.6 mW/mm² for 100 ms (blue bar). (FIG. 9C) Absorption, excitation, and emission spectra of rScarlet.

FIG. 10 provides graphs of results from evaluation of TMI for subcellular labeling with four red FPs. The same experiments as in FIG. 7 were run for the four red FPs used in TMI and two sets of Pearson's correlation values were obtained for each red FP. Bar plots of mean with SD are used, with individual values plotted as dots; n=5 to 6 images from two culture batches. Unpaired t test was run between two sets of Pearson's correlation values for each FP; n.s., no significance.

FIG. 11A-B provides images of simulation of temporally multiplexed imaging of four red FPs. A 70-frame time lapse image was generated using a pre-acquired fluorescence image of NIH/3T3 cells (same image as used in FIG. 8) and the off-switching traces in FIG. 3B. The fluorescence components of the four red FPs were randomly assigned and mixed. Different cells have different fluorescence combinations of the four red FPs. Poisson noise was added to each pixel to mimic real images. (FIG. 11A) Simulated movie showing cells that express four red FPs. (FIG. 11B) After signal unmixing, four images were obtained for each FP. The images were then compared with ground-truth images. Pearson's correlation values between unmixed images and ground-truth images are shown. Detailed value comparisons are shown in FIG. 3F.

FIG. 12A-B provides images of single-color “brainbow” in zebrafish. (FIG. 12A) Representative raw fluorescent images of zebrafish larvae in spinal cord and forebrain. (FIG. 12B) Brainbow-like images showing the same area in FIG. 12A. Scale bars, 20 μm.

FIG. 13 provides a graph illustrating CDK2 activity progression during consecutive and complete G1 and S phase. Data are shown as mean±SD with all individual values plotted. The amplitudes of CDK2 activity increased from middle G1 phase to early S phase is larger than that increased from early S phase to late S phase, n=10 cells from 5 culture batches. Wilcoxon rank sum tests, **p<0.01.

### DETAILED DESCRIPTION

According to an aspect of the invention, methods and systems are provided in which fluorophores are associated with different clocklike temporal properties, with different genes, so that they can all be imaged at the same time, and then sorted out through linear unmixing. In some embodiments, reversibly photoswitchable fluorescent proteins (rsFPs) are used in methods of the invention, resulting in different off rates to indicate the expression of different genes. This permits the signals to be separated out through standard linear algebra. As set forth herein, methods of the invention, referred to herein as temporally multiplexed imaging (TMI), may be used to measure cell activities, a non-limiting example of which are cell kinase activities. As used herein the term “measure” means to determine. TMI requires no hardware beyond standard epifluorescent or confocal microscopes, and indeed, can image many signals at once, even using a single-color channel, because the information is encoded in time, not through the spectrum.

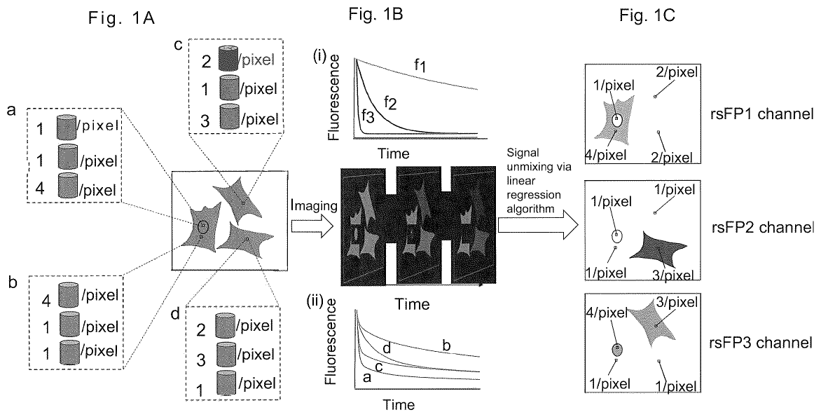

In the initial implementation of TMI, it was determined that multiple rsFPs, even of the same color (i.e. similar spectral properties), but different clocklike or temporal behaviors (e.g. different off-switching rates during continuous imaging) could be expressed in the same cell (FIG. 1A, FIG. 1Bi). Because each fluorophore behaves differently over time during continuous imaging (e.g., exhibits distinct fluorescence traces as a function of time), different pixels exhibiting different combinations of fluorophores, were found to exhibit different time courses of fluorescence (FIG. 1Bii). By computationally unmixing the fluorescence trace into a linear combination of the reference traces exhibited by each fluorophore alone (i.e., in a separate experiment under the same imaging conditions), it was possible to reconstruct the image of each fluorophore by assigning the linear weight associated with a given fluorophore, to each pixel of the image (FIG. 1C).

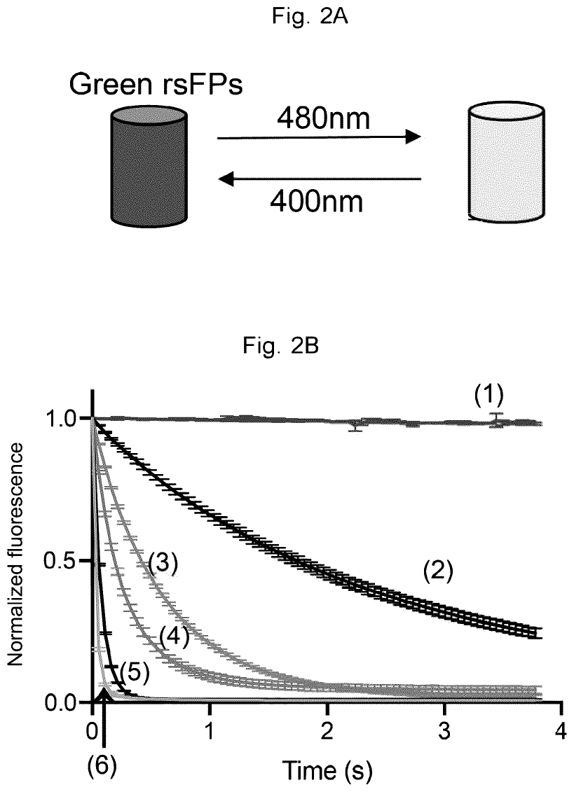

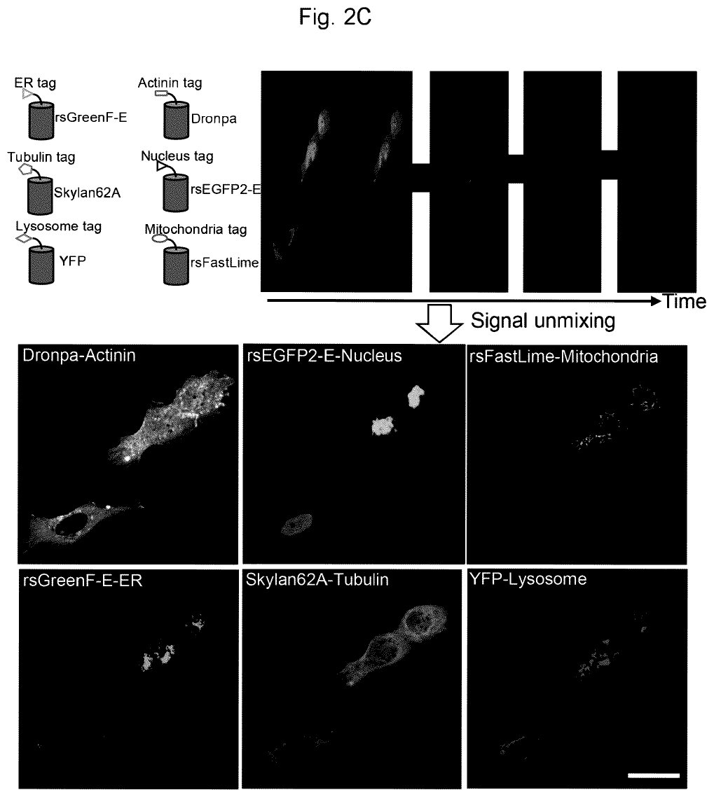

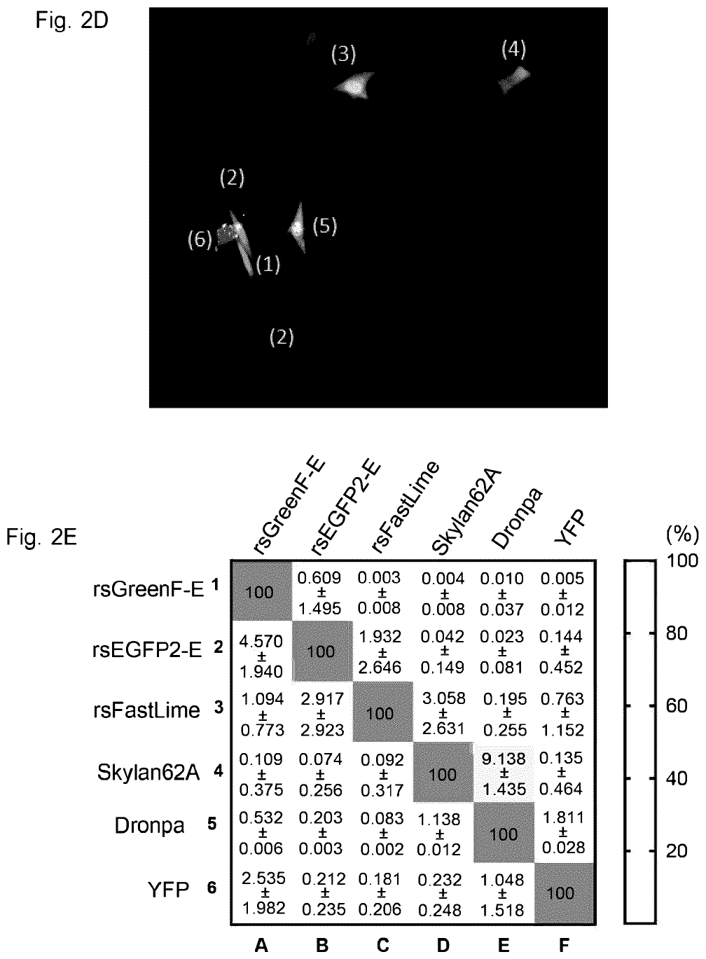

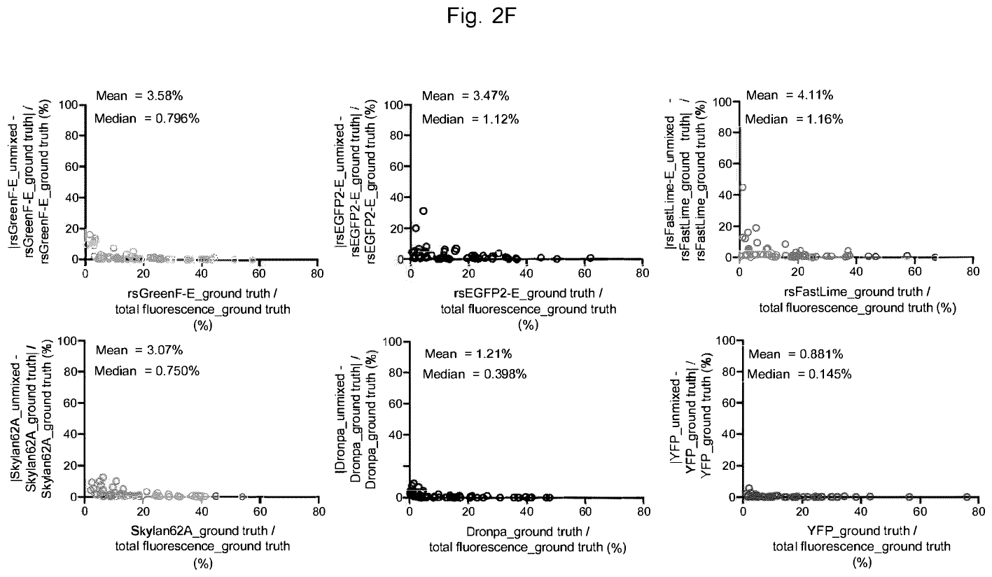

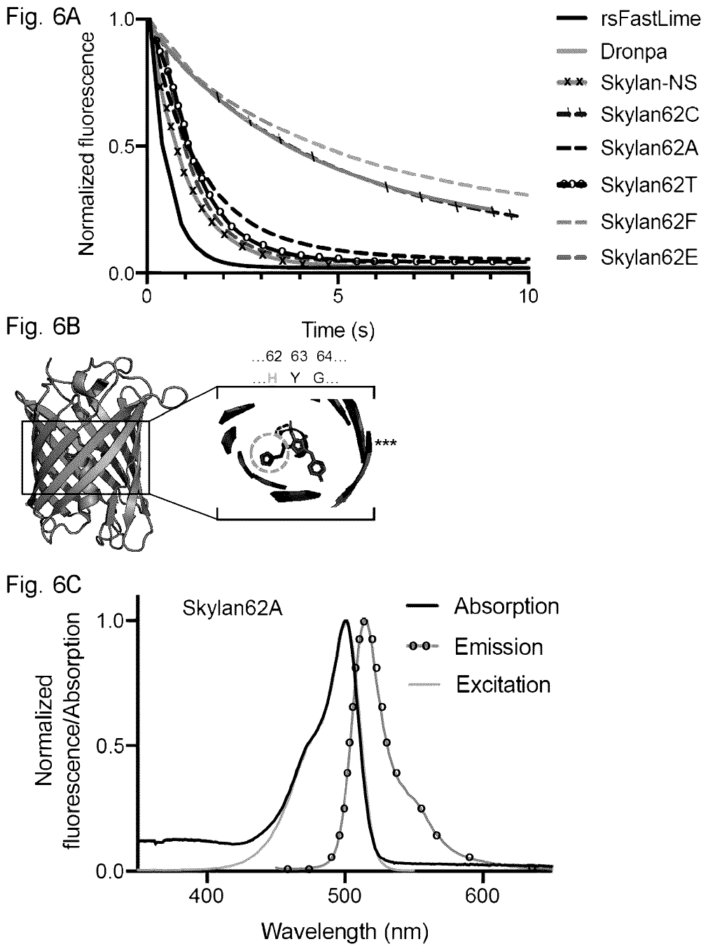

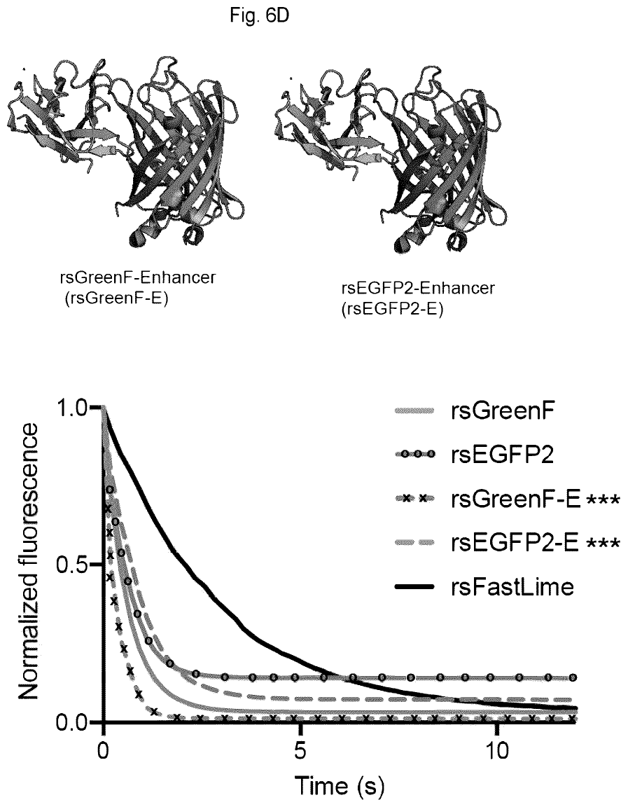

To validate this concept, studies were performed to explore green rsFPs, such as those whose fluorescence can be switched “off” by blue/cyan light and switched “on” by purple light (FIG. 2A). Initial studies were performed to determine a set of green rsFPs with distinct off-switching kinetics, so that they could be distinguished in TMI imaging of cells (FIG. 2B). It was determined that Dronpa (Ando R, et al., Science, (2004); 306(5700):1370-3), rsFastLime (Stiel A C, et al., Biochem J., (2007); 402(1):35-42), and rsGreenF-Enhancer (rsGreenF-E) (Roebroek T, et al., Int J Mol Sci [Internet], (2017); 18(9)) exhibited extremely different off-switching kinetics (FIG. 2B). Skylan-NS (Zhang X, et al., Proc Natl Acad Sci USA, (2016); 113(37):10364-9) had kinetics similar to rsFastLime (FIG. 6A), so directed evolution was performed (FIG. 6A-B) to identify a variant that was then named Skylan62A (Skylan-NS-L62A), that exhibited photoswitching kinetics between rsFastLime and Dronpa (FIG. 6B), without changing spectral characteristics (FIG. 6C). rsEGFP2-Enhancer (rsEGFP2-E) ((Roebroek T, et al., Int J Mol Sci [Internet], (2017); 18(9)), (Grotjohann T, et al., Elife, (2012); 1:e00248)) was identified as an rsFP with off-switching kinetics between rsGreen-F-E and rsFastLime (FIG. 6D). Finally, a non-switching fluorescent protein, YFP (Ormö M, et al., Science, (1996); 273(5280):1392-5) was added to create a 6th temporally distinguishable candidate (FIG. 2B).

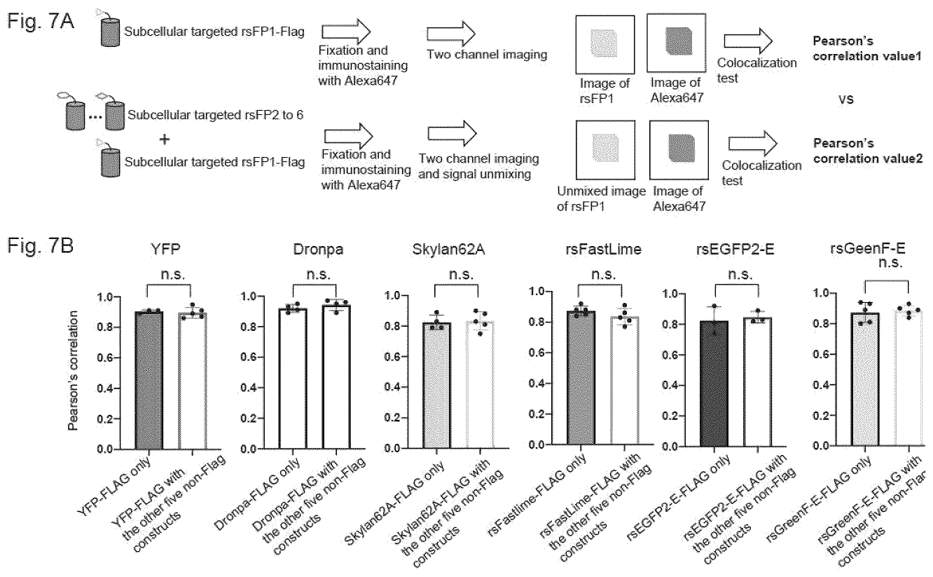

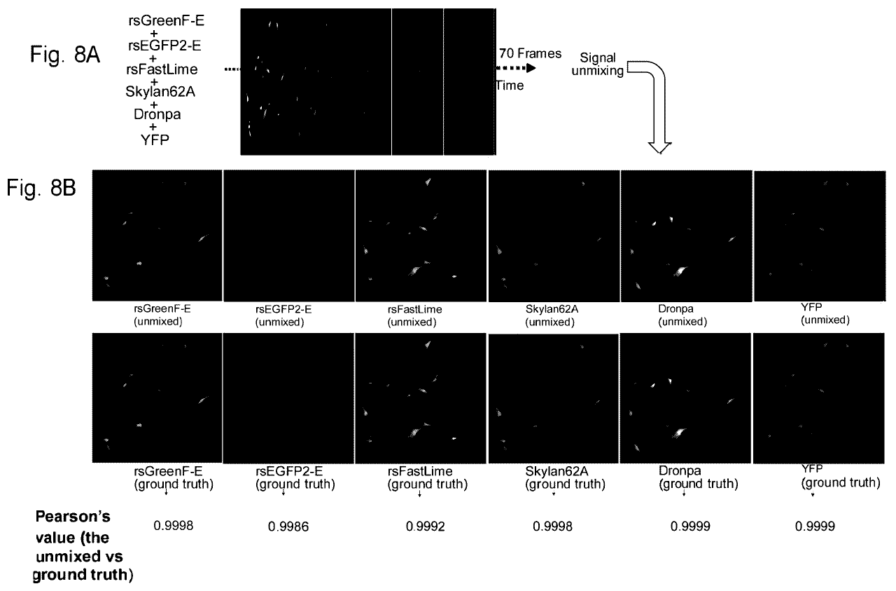

The six aforementioned rsFPs were expressed in U2OS cells, each fused to a distinct, well-validated, subcellular targeting sequence so that each fluorophore would be targeted to a different biological structure (FIG. 2C). The cells were fixed and 70-frame movies were acquired over 3.8 seconds, on a standard confocal microscope. The movies were then unmixed using the reference traces (FIG. 2B) using standard linear unmixing algebra (see Methods in Examples Section). As a first strategy for validating the unmixing, the resulting images were compared to those obtained by antibody staining against a FLAG-tag fused to each of the six FPs in turn, and no difference in the correlation between the rsFPs and the antibody staining was found when the rsFPs were expressed individually (which serves as ground truth), vs. when they were expressed all at the same time (FIG. 7A-B). Thus, crosstalk between different rsFPs is minimal when analyzed in this temporally multiplexed way. As a second measure of TMI crosstalk, they were expressed in individual NIH/3T3 cells, with each cell expressing only one FP, to serve as ground truth, and then the crosstalk of each FP to the other five FPs (FIG. 2D) was measured, and it was determined that crosstalk was in the few percent range, and often much lower (FIG. 2E). Finally, as a third, independent, validation, we simulated a 70-frame video based on a fluorescence image of NIH/3T3 cells, with each pixel exhibiting a linear combination of the six fluorophores of FIG. 2B (FIG. 8A), thus serving as a ground truth. Simulated movies of the fluorescence dynamics were prepared, including added noise, and then the movies were unmixed as above to obtain the reconstructed single-fluorophore images (FIG. 8B). Near-perfect Pearson's correlation values between unmixed images and ground truth images were obtained—exceeding 99.8% (FIG. 8B). Analysis of individual cells (FIG. 2F) showed that most cells had deviations from the ground truth of a few percent, although for extremely dim cells, the error, as expected, could be greater. Thus, in several independent validations, it was determined that linear unmixing of summed fluorescence movies of cells containing mixtures of fluorophores yielded highly accurate reconstructions of the data when compared to ground truth datasets or simulations.

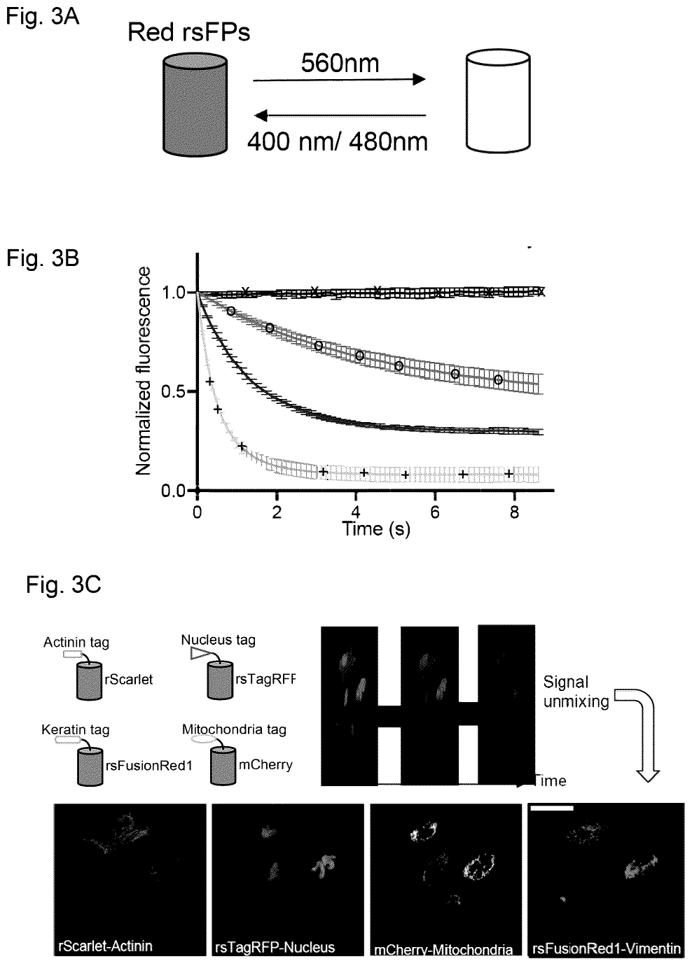

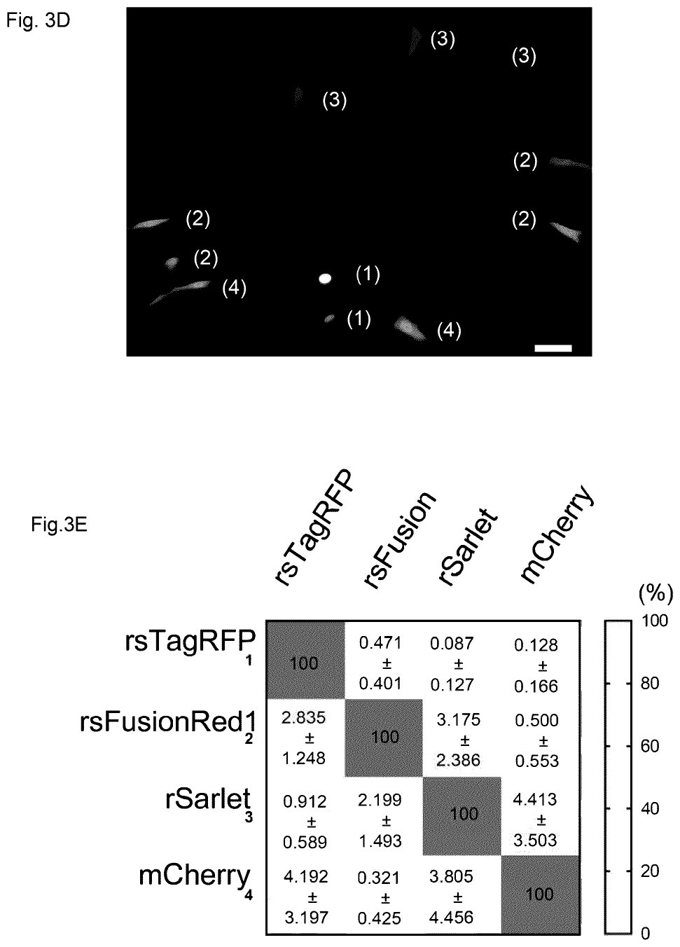

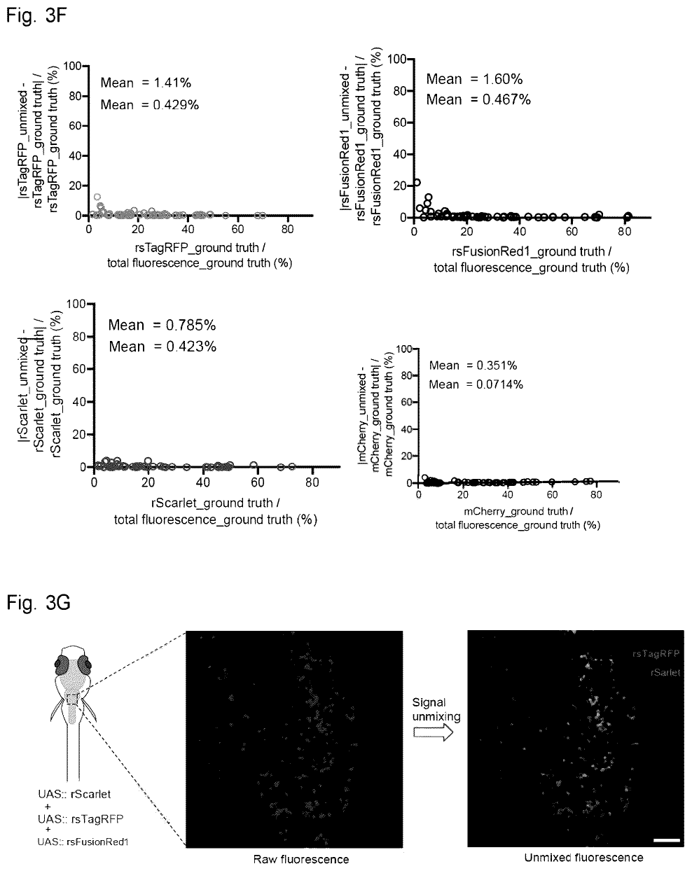

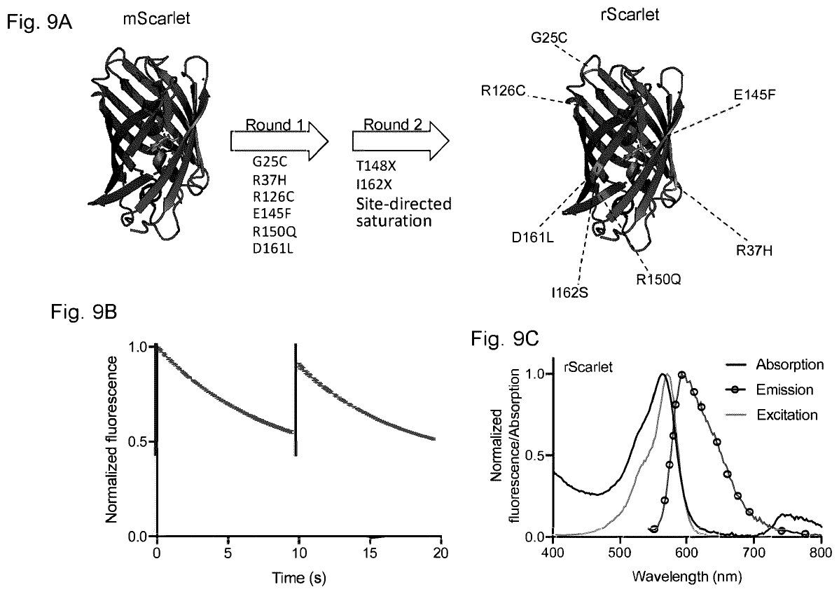

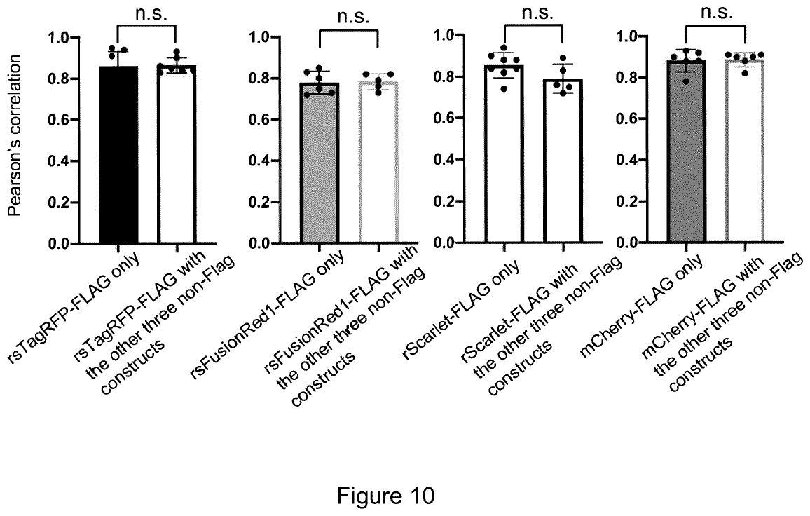

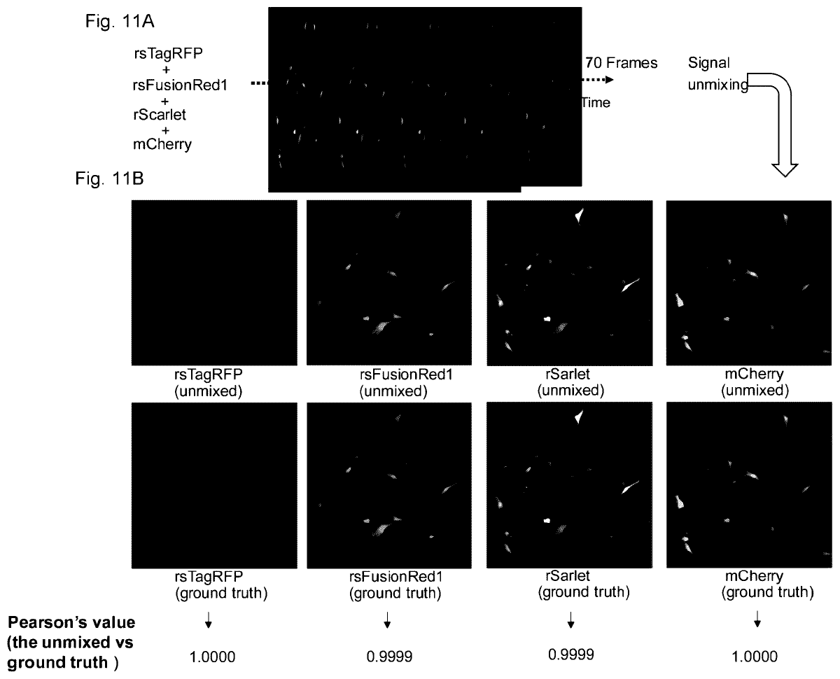

Studies were conducted to explore the use of off-switching red rsFPs for TMI. Off-switching red rsFPs are a type of FPs whose fluorescence can be switched to a dim state when illuminated with yellow/orange light and will resume bright fluorescence under illumination by blue/cyan or purple light (FIG. 3A). rsTagRFP (Pletnev S, et al., J Mol Biol., (2012); 417(3):144-51) and rsFusionRed1 (Pennacchietti F, et al., Nat Methods, (2018); 15(8):601-4) were selected as two candidates for TMI, based on their switching rates (FIG. 3B). A new rsFP was engineered and named rScarlet, from mScarlet (Bindels D S, et al., Nat Methods, (2017); 14(1):53-6), inspired by the development of rsCherryRev1.4 from mCherry ((Stiel A C, et al., Biophys J., (2008); 95(6):2989-97), (Lavoie-Cardinal F, et al., Chemphyschem, (2014); 15(4):655-63)) (FIG. 9A). rScarlet exhibited slower photoswitching kinetics than rsTagRFP and rsFusionRed1, and similar spectral properties to mScarlet (FIG. 9B-C, FIG. 3B). Next, a non-switching RFP, mCherry (Shaner N C, et al., Nat Biotechnol., (2004); 22(12):1567-72) was selected as the fourth fluorophore for temporal multiplexing. Many other commonly used RFPs exhibited substantial off-photoswitching with excitation by yellow/orange light. The four red FPs were expressed in U2OS cells, with each FP targeting different subcellular structures, the cells were fixed, and 70-frame movies were acquired over 8.6 seconds, on a standard confocal microscope. After linear unmixing, each unmixed image showed the expected subcellular structures (FIG. 3C), with minimal crosstalk when validated with antibody staining against epitope-tagged indicator (FIG. 10). When the four FPs were expressed in NIH/3T3 cells individually, the crosstalk between each pair of FPs was in the few percent range (FIG. 3D-E). Repeating the simulation exercise revealed that, as with green rsFPs, red rsFPs could yield extremely low crosstalk (FIG. 3F, FIG. 11). As with green rsFPs, red rsFPs could support, as characterized in multiple ways, well-separable signals with low crosstalk.

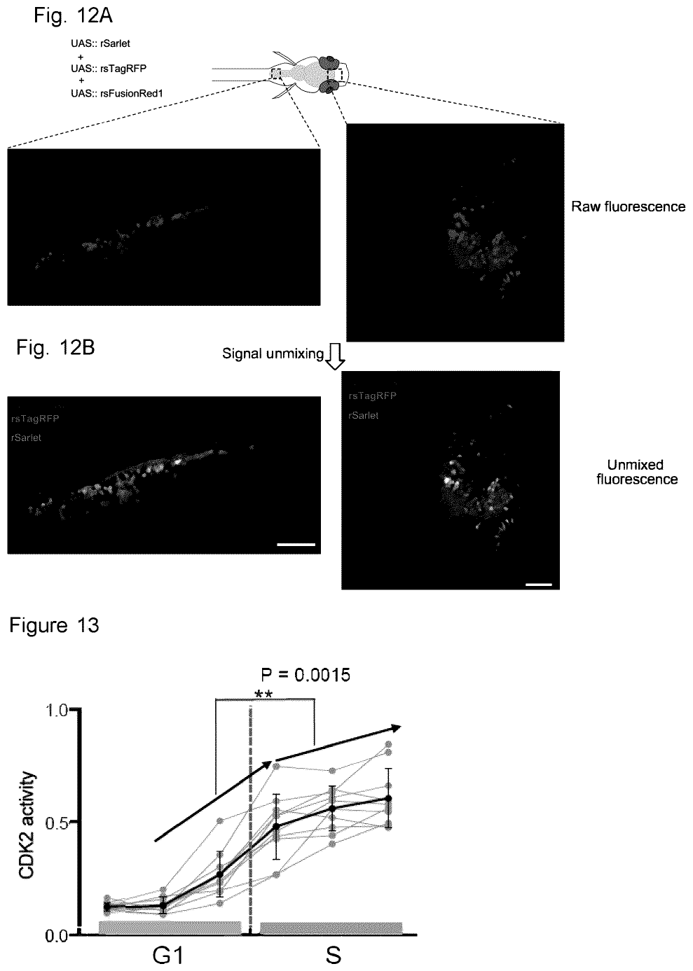

Brainbow, the combinatorial expression of fluorescent proteins in neurons for neural identification and tracing (Livet J, et al., Nature, (2007); 450(7166):56-62), is popular but requires multispectral imaging. Studies were performed to determine whether TMI could support a “single color brainbow” strategy. Studies included transiently expressing rsTagRFP, rsFusionRed1, and rScarlet in the nervous system of zebrafish larvae. Transient expression of a gene of interest in zebrafish via plasmid injection is in a mosaic manner (Köster R W, and Fraser S E, Dev Biol., (2001); 233(2):329-46). Previous studies have shown that mosaic expression of two genes in zebrafish exhibited very different patterns even under the same promoter ((Köster R W, and Fraser S E, Dev Biol., (2001); 233(2):329-46), (Formella I, et al., Redox Biol., (2018); 19:226-34)). Studies were performed to determine whether mosaic expression of three FPs in zebrafish with different patterns would achieve a similar gene expression effect as what cre-lox recombination does. Indeed, different expression patterns were observed from the three FPs and brainbow-like images were obtained in living zebrafish brain and spinal cord via TMI (FIG. 3G, FIG. 12). Superior to traditional brainbow technology, TMI-based brainbow only occupies one imaging channel and frees up optical channels for other purposes such as imaging of Ca²⁺, membrane potential, and neurotransmitters.

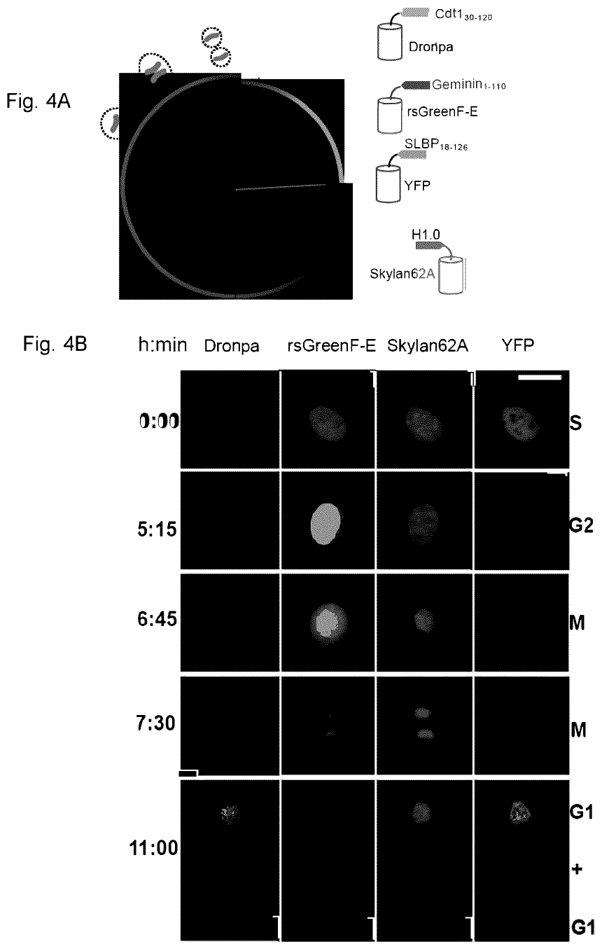

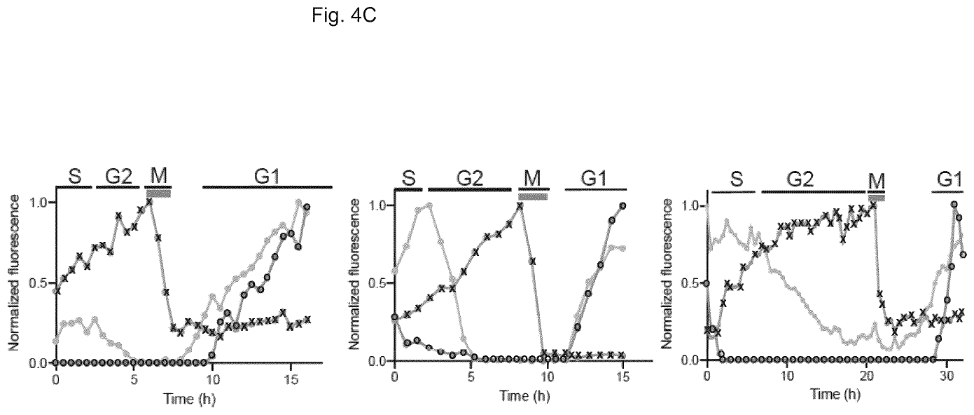

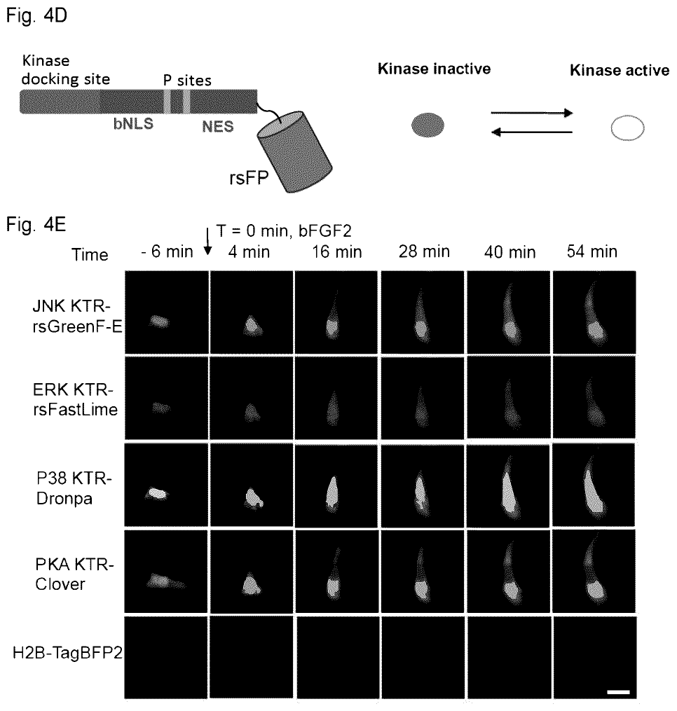

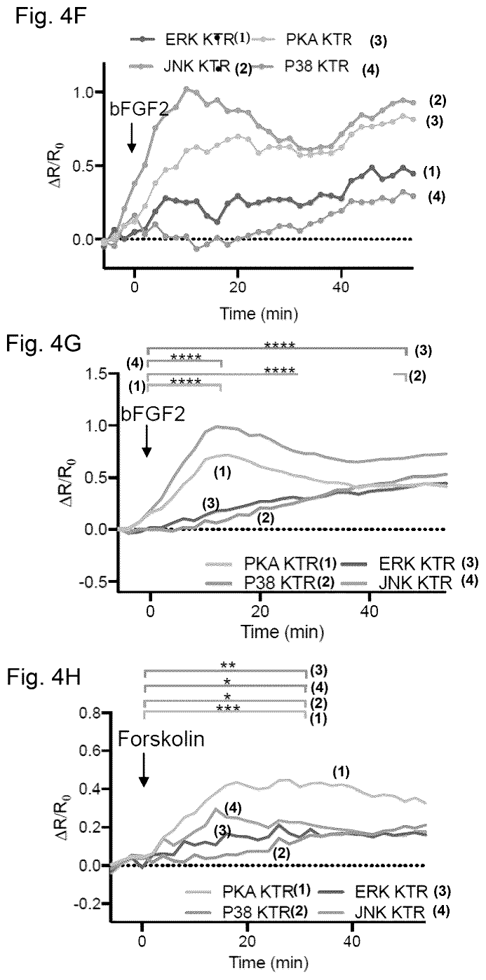

Studies were then performed to explore whether TMI could help with the visualization of cellular dynamics in living cells. FUCCI4 is an indicator system that reports all four cell cycle phases based on cell cycle-regulated proteins fused to spectrally distinct FPs (Bajar B T, et al., Nat Methods, (2016); 13(12):993-6). Here, a single-color version of FUCCI4 was developed by replacing the four FPs in the original FUCCI4 with four of the six green FPs that were used for TMI (FIG. 4A). Specifically, Dronpa, YFP, rsGreenF-E, and Skylan62A were fused to the cell cycle-regulated proteins Cdt130-120, SLBP18-126, Geminin1-110, and histone H1.0 respectively. Analogous to the original FUCCI4, the G1-S transition is reported by the emergence of rsGreenF-E fluorescence while YFP fluorescence persisted, and the S-G2 transition was marked by the loss of YFP and stable rsGreenF-E fluorescence. Chromosome condensation, as reported by Skylan62A, indicates the M phase; finally, loss of rsGreenF-E fluorescence and the appearance of Dronpa and YFP fluorescence means the beginning of the G1 phase. Cell cycle transitions were imaged in NIH/3T3 cells using the single-color FUCCI4 system and results demonstrated the ability to identify each transition in the cell cycle just as with the original FUCCI4 (FIG. 4B-C).

Studies were then performed to explore whether TMI could help with the imaging of signals other than gene expression. Kinase translocation reporters (KTRs) are fluorescent sensors that report protein phosphorylation by translocating between the cytoplasm and the nucleus (Regot S, et al., Cell, (2014); 157(7):1724-34) (FIG. 4D, left). When the kinase of interest is inactive, the KTR is unphosphorylated, which leads its nuclear localization; when the kinase of interest is active, phosphorylation of the KTR leads translocation of sequences to cause cytoplasmic localization (FIG. 4D, right). The FPs in the original KTRs were replaced for JNK, ERK, and P38, with rsGreenF-E, rsFastLime, and Dronpa respectively. Together with PKA KTR-Clover (Clover being a non-photoswitching FP), there were four kinase sensors that could be used simultaneously via TMI. NIH/3T3 cells expressing all four KTRs and H2B-TagBFP as a nuclear marker were imaged and stimulated with mouse basic fibroblast growth factor 2 (bFGF2, 20 ng/ml), which is known to drive kinases such as JNK, P38, ERK, and PKA [(Pursiheimo J P, et al., Proc Natl Acad Sci USA, (2000); 97(1):168-73), (Lichtenstein M P, et al., Neurosignals, (2012); 20(2):86-102), (Kim B S, et al., Biochem Biophys Res Commun., (2014); 450(4):1333-8), (Kanazawa S, et al., PLoS One, (2010); 5(8):e12228)]. Fast onset of the JNK KTR signal was observed, as well as the PKA KTR signal, both of which tapered off somewhat over time, whereas the ERK and P38 KTR signals steadily increased over time (FIG. 4F-G). In contrast, forskolin preferentially induced PKA activity, with lesser activity for the other three kinases, as compared to the bFGF2 case (FIG. 4H), consistent with what has been observed previously [(Mehta S, et al., Nat Cell Biol, (2018); 20(10):1215-25), (Park K H, et al., Toxicol Sci., (2012); 128(1):247-57), (Delghandi M P, et al., Cell Signal, (2005); 17(11):1343-51)].

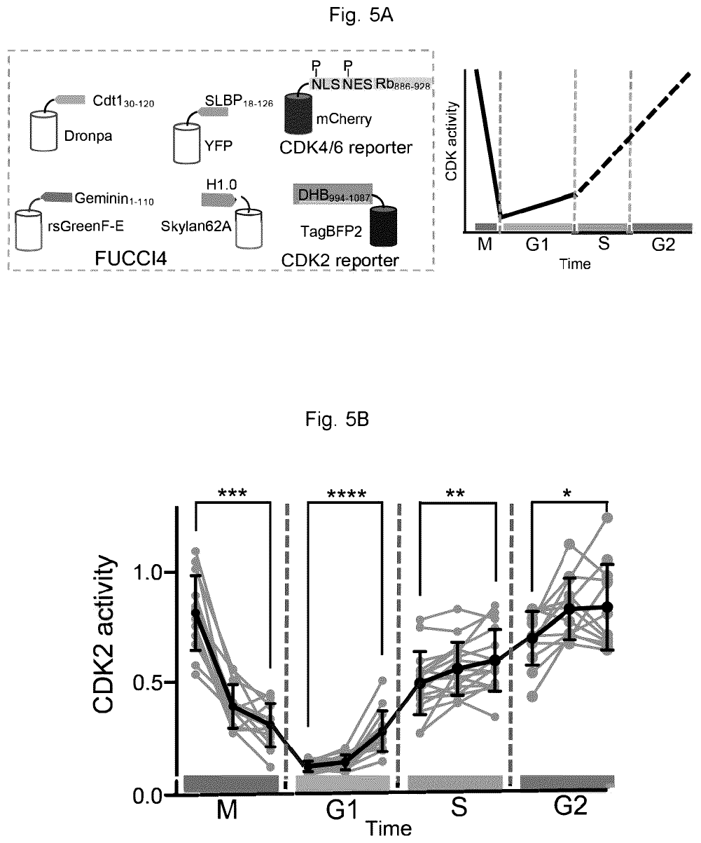

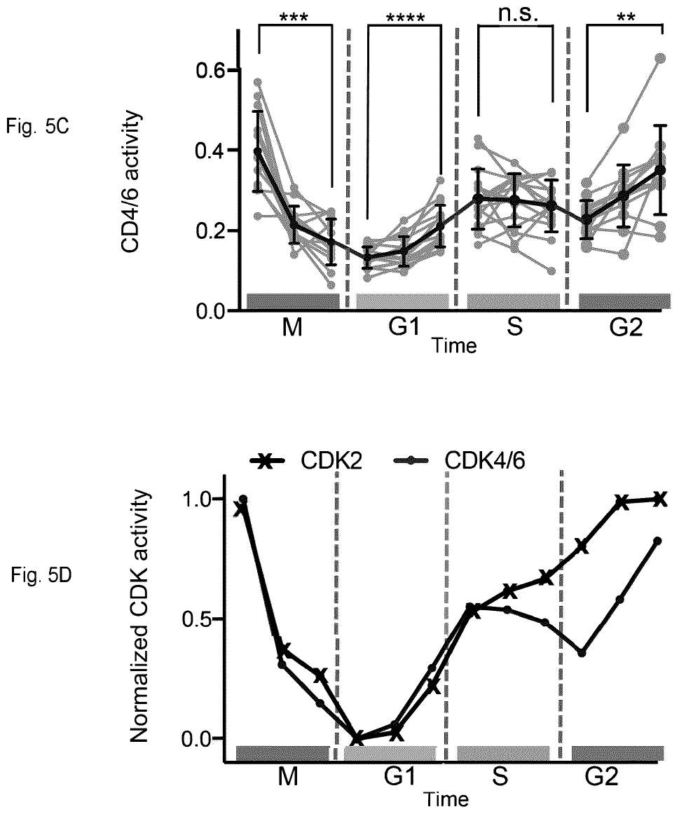

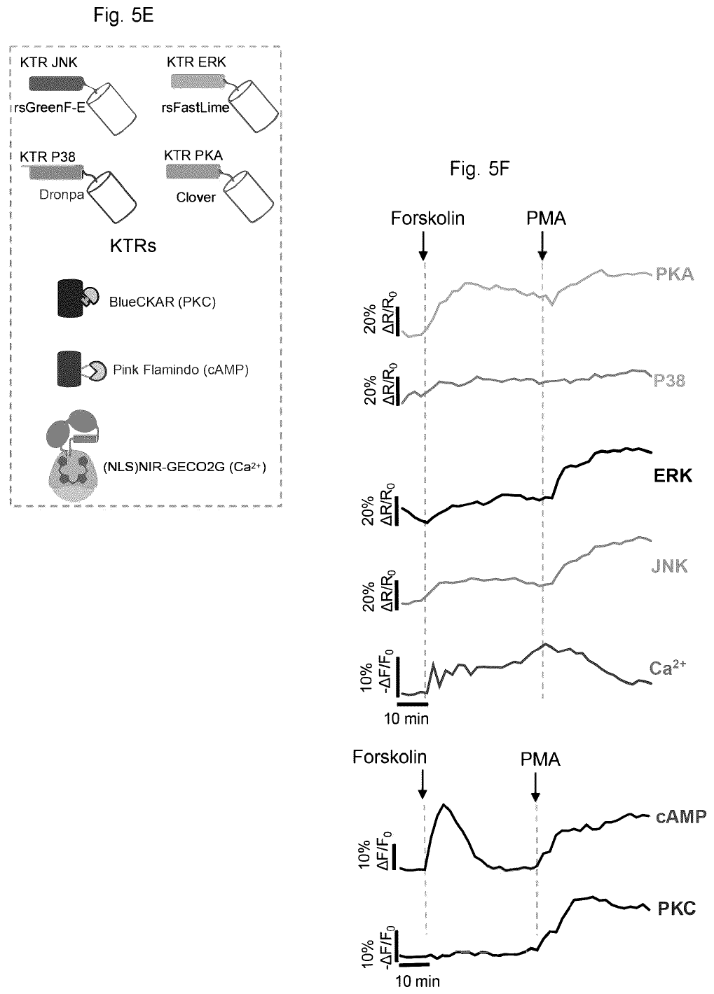

Embodiments of TMI methods of the invention were shown to enable monitoring of many expressed genes at once (FIG. 3G, FIG. 4A-C) or many dynamical reporters at once (FIG. 4D-H), even using a single-color channel. Further studies were performed to determine whether this advantage could be used to image more signals in a living cell than had been simultaneously measured using fully genetically encoded probes, on a conventional microscope—the key to deployability in everyday biology. As an example of the kind of experiment that can be done, a TMI indicator set was combined with conventional spectral multiplexing. First, single-color FUCCI4 was combined with conventional reporters of cyclin-dependent kinases 2 (CDK2, based on TagBFP2) (Spencer S L, et al., Cell, (2013); 155(2):369-83) and 4/6 (CDK4/6, based on mCherry) (Yang H W, et al., Elife [Internet], (2020); 9) (FIG. 5A, left). As with the kinase imaging. The two CDK reporters stayed in the cell nucleus when their respective kinases were inactive and translocated to the cell cytoplasm when activated. Cell cycle progression is cooperatively regulated by multiple CDKs, knowing how CDKs activities proceed in each cell cycle phase is critical to understanding the molecular mechanisms underlying cell cycle regulations. Past results indicate that both CDK2 and CDK4/6 enter the M phase with high-level activities, which drop rapidly during mitosis through to the early G1 phase, and then start to build up in the G1 phase (FIG. 5A, right) [(Spencer S L, et al., Cell, (2013); 155(2):369-83), (Yang H W, et al., Elife [Internet], (2020); 9), (Stern B, and Nurse P., Trends Genet., (1996); 12(9):345-50), (Coudreuse D, and Nurse P., Nature, (2010); 468(7327):1074-9]). However, even though it was shown that both CDK2 and CDK4/6 activities trend up from the start of the S phase to the end of the G2 phase [(Spencer S L, et al., Cell, (2013); 155(2):369-83), (Yang H W, et al., Elife [Internet], (2020); 9)], how the amplitudes of CDK2 and CDK4/6 activities change during the S and G2 phase were unknown. Here, with use of embodiments of the invention comprising TMI-based FUCCI4, it was possible to observe how CDK2 and CDK4/6 activities changed in all four cell cycle phases including the S phase and G2 phase in NIH/3T3 cells.

To facilitate the analysis of CDK activity traces from a population of cells with various lengths of the same cell cycle phases, each cell cycle phase was divided into early, middle, and late stages with even durations, and the CDK activity in a given stage was obtained by averaging all CDK activity values in that stage. Thus, all CDK traces of each cell cycle phase were normalized to simpler traces with only three data points regardless of cell cycle phase durations (FIG. 5B-C). Results showed that high-level CDK2 and CDK4/6 activity went through an abrupt drop in the M phase, reached the bottom in the early G1 phase, and then started to build up from low level with the G1 phase proceeding (FIG. 5B-C). These results are consistent with what was observed previously [(Spencer S L, et al., Cell, (2013); 155(2):369-83), (Yang H W, et al., Elife [Internet], (2020); 9]). In addition, it was determined that after entering the S phase, CDK2 activity kept rising at a slow rate (the rising level from the early S phase to the late S phase was smaller than that from the middle G1 phase to the early phase(FIG. 5B, FIG. 13)); the rise continued during S/G2 transition until reaching a plateau in middle/late G2 phase followed by M phase entry (FIG. 5B, D). Different from CDK2, CDK4/6 activity reached a plateau in the early S phase throughout the end of the S phase, followed by an increase in the whole G2 phase before entering the M phase (FIG. 5C, D). These results revealed how CDK2 and CDK4/6 activity accumulates in S and G2 phases respectively for the first time in mammalian cells and can provide new insights into the molecular mechanisms underlying cell cycle regulations.

Studies were then performed in which green FPs-based KTRs were combined with NIR-GECO2G (Qian Y, et al., PLoS Biol., (2020); 18(11):e3000965), Pink Flamindo (Harada K, et al., Sci Rep., (2017); 7(1):7351), and BlueCKAR (Mehta S, et al., Nat Cell Biol, (2018); 20(10):1215-25) with the goal of simultaneously observing the following seven cellular dynamics within single cells: JNK, ERK, P38, PKA, Ca²⁺, cAMP, and PKC (FIG. 5E). NIH/3T3 cells with the expression of all seven reporters were imaged before and after the stimulation of 50 μM forskolin and 100 ng/ml phorbol 12-myristate 13-acetate (PMA). Forskolin induces substantial cAMP, Ca²⁺, and PKA response, lesser ERK, P38, and JNK response, and no PKC response as has been demonstrated [(Linghu C, et al., Cell, (2020); 183(6):1682-98.e24), (Mehta S, et al., Nat Cell Biol, (2018); 20(10):1215-25)]. Results showed an obvious increases in PKA, Ca²⁺, and cAMP activity after treatment of forskolin while ERK, p38, and JNK showed little elevations and no substantial response from PKC (FIG. 5F). PMA is a commonly used PKC activator, but studies have shown that it could also effectively activate PKA, cAMP, JNK, and ERK [(Baillie G, et al., Mol Pharmacol., (2001); 60(5):1100-11), (Sriraman V, et al., Mol Cell Endocrinol., (2008); 294(1-2):52-60)]. Indeed, after administration of PMA, PKC, PKA, cAMP, JNK and ERK exhibited strong responses while P38 and Ca²⁺ remained little to no changes (FIG. 5F). Thus, by combining spectral and temporal multiplexing it was possible to image unprecedented seven different signals from individual cells at once.

TMI methods of the invention provide an accurate, versatile, and easily adaptable imaging technology by virtue of distinct temporal behaviors of a set of fluorescent proteins. It is suitable for both functional and structural imaging of live or fixed samples on standard epifluorescence or confocal microscopes without any hardware equipment upgrades.

As demonstrated experimentally, methods of the invention made it possible to observe large numbers of cellular signals by using only one optical channel via TMI, which means increased numbers of cellular signals could be imaged at once when two or more optical channels are used. With that, the activity of CDK2 and CDK4/6 were examined at once in all four cell cycle phases and results demonstrated it was possible to simultaneously observe seven cell signals in single cells, both of which could not be achieved without TMI.

It was determined that TMI methods of the invention rely on repeatable photoswitching behaviors of rsFPs, thus even illumination across field-of-views (which could be adjusted using a homogenously fluorescent sample) is important for accurate signal unmixing. Second, due to pixel-wide image processing, artificial movements of imaging samples during each cycle of photoswitching should be avoided. Otherwise, image registration might be needed. Third, the imaging time for each round of photoswitching ranges from 3 s to 16 s in the experiments depending on the illumination intensity. But stronger excitation could be used if a shorter time (such as <1 s) may be used in methods of the invention. Fourth, in order to obtain sufficient temporal information for subsequent signal unmixing, some embodiments of methods of the invention comprise acquiring a 50 (or up)-frame movie for each round of photoswitching, with the intensity of Dronpa dropping to 35% (or less) of its original fluorescence at the end for multiplexing of green FPs and the intensity of rScarlet dropping to 60% (or less) of its original fluorescence at the end for multiplexing of red FPs. Fifth, even though rsFPs could resume their fluorescence with minimal losses after each round of off-switching [(Ando R, et al., Science, (2004); 306(5700):1370-3), (Stiel A C, et al., Biochem J., (2007); 402(1):35-42), (Roebroek T, et al., Int J Mol Sci [Internet], (2017); 18(9)), (Pletnev S, et al., J Mol Biol., (2012); 417(3):144-51), (Pennacchietti F, et al., Nat Methods, (2018); 15(8):601-4)], the photobleaching in TMI is larger than that in conventional imaging due to prolonged illumination when recording continuous signals with short-time intervals (seconds to minutes).

TMI methods of the invention may be used to examine and/or optimize the signal unmixing algorithm for higher accuracy and faster computing and improve the brightness of currently available rsFPs, most of which are dimmer than commonly used non-switching FPs (Table 1). Details of statistical analysis used to prepare various figures provided herein are provided in Table 2. It will be understood that methods of the invention can be used with alternative sets of photoswitchable fluorescent dyes, non-limiting examples of which are bright photoswitchable fluorescent dyes, in addition to those set forth herein. Thus, TMI methods of the invention can provide solutions for higher-order multiplexed immunofluorescence imaging of clear/expanded samples.

TABLE 1
Spectroscopic characteristics of FPs
used for TMI in certain embodiments.

| | | | | | | Switch- |
|---|---|---|---|---|---|---|
| | Ex | Em | EC | | | off half |
| | max | max | (×10³ | | | time |
| FP | (nm) | (nm) | M⁻¹cm⁻¹) | QY | Brightness | (s)^{j} |
| rsGreenF-E^{a} | 485 | 511 | 66 | 0.39 | 25.74 | 0.02273 |
| rsEGFP2-E^{b} | 493 | 510 | 47 | 0.36 | 16.92 | 0.05233 |
| rsFastLime^{c} | 496 | 518 | 39 | 0.77 | 30.03 | 0.1961 |
| Skylan62A | 502 | 514 | 84 | 0.61 | 51.53 | 0.4189 |
| Dronpa^{d} | 503 | 518 | 95 | 0.85 | 80.75 | 1.546 |
| YFP^{e} | 513 | 527 | 67 | 0.67 | 44.89 | N/A |
| Clover^{f} | 505 | 515 | 111 | 0.67 | 84.36 | N/A |
| rsTagRFP^{g} | 567 | 585 | 37 | 0.11 | 4.05 | 0.3928 |
| rsFusionRed1^{h} | 577 | 605 | 82 | 0.1 | 8.24 | 0.9587 |
| rScarlet | 570 | 592 | 61 | 0.44 | 26.97 | 3.035 |
| mCherryⁱ | 587 | 610 | 72 | 0.22 | 15.84 | N/A |

Abbreviations: Ex max, fluorescence excitation maximum; Em max, fluorescence emission maximum; EC, extinction coefficient; QY, quantum yield. Brightness is defined as the product of EC and QY.
^{a}Data from Roebroek T, et al., Int J Mol Sci [Internet], (2017); 18(9);
^{b}data from Grotjohann T, et al., Elife, (2012); 1: e00248;
^{c}data from Stiel A C, et al., Biochem J., (2007); 402(1): 35-42;
^{d}data from r Ando R, et al., Science, (2004); 306(5700): 1370-3;
^{e}data from Ormö M, et al., Science, (1996); 273(5280): 1392-5;
^{f}data from Lam A J, et al., Nat Methods, (2012); 9(10): 1005-12;
^{g}data from Pletnev S, et al., J Mol Biol., (2012); 417(3): 144-51;
^{h}data from Pennacchietti F, et al., Nat Methods, (2018); 15(8): 601-4;
ⁱdata from Shaner N C, et al., Nat Biotechnol., (2004); 22(12): 1567-72;
^{j}Measured in NIH/3T3 cells under continuous illumination (green rsFPs: 488 nm at 40 mW/mm²; red rsFPs: 561 nm at 50 mW/mm²).
N/A, not applicable.

TABLE 2
Statistical analysis for indicated figures.
Statistical analysis for FIG. 4G
Wilcoxon rank sum test of PKA activity before (averaged value from t = −6 min to t = 0
min) and after FGF2 treatment (averaged value from t = 12 min to t = 18 min)

| | |
|---|---|
| P value | 0.00002642 |
| Sum Rank (before FGF2 treatment) | 152 |
| Sum Rank (after FGF2 treatment) | 376 |
| Z value | −4.20231 |

Wilcoxon rank sum test of P38 activity before (averaged value from t = −6 min to t = 0
min) and after FGF2 treatment (averaged value from t = 48 min to t = 54 min)

| | |
|---|---|
| P value | 0.0000508776 |
| Sum Rank (before FGF2 treatment) | 156 |
| Sum Rank (after FGF2 treatment) | 372 |
| Z value | −4.05156 |

Wilcoxon rank sum test of ERK activity before (averaged value from t = −6 min to t = 0
min) and after FGF2 treatment (averaged value from t = 48 min to t = 54 min)

| | |
|---|---|
| P value | 0.0000134244 |
| Sum Rank (before FGF2 treatment) | 148 |
| Sum Rank (after FGF2 treatment) | 380 |
| Z value | −4.35307 |

Wilcoxon rank sum test of JNK activity before (averaged value from t = −6 min to t = 0
min) and after FGF2 treatment (averaged value from t = 12 min to t = 18 min)

| | |
|---|---|
| P value | 0.0000432775 |
| Sum Rank (before FGF2 treatment) | 155 |
| Sum Rank (after FGF2 treatment) | 373 |
| Z value | −4.08925 |

Statistical analysis for FIG. 4H
Wilcoxon rank sum test of PKA activity before (averaged value from t = −6 min to t = 0
min) and after forskolin treatment (averaged value from t = 30 min to t = 36 min)

| | |
|---|---|
| P value | 0.000329839 |
| Sum Rank (before forskolin treatment) | 57 |
| Sum Rank (after forskolin treatment) | 153 |
| Z value | −3.59066 |

Wilcoxon rank sum test of P38 activity before (averaged value from t = −6 min to t = 0
min) and after forskolin treatment (average value from t = 30 min to t = 36 min)

| | |
|---|---|
| P value | 0.01402 |
| Sum Rank (before forskolin treatment) | 72 |
| Sum Rank (after forskolin treatment) | 138 |
| Z value | −2.45677 |

Wilcoxon rank sum test of ERK activity before (averaged value from t = −6 min to t = 0
min) and after forskolin treatment (averaged value from t = 30 min to t = 36 min)

| | |
|---|---|
| P value | 0.00361 |
| Sum Rank (before forskolin treatment) | 66 |
| Sum Rank (after forskolin treatment) | 144 |
| Z value | −2.91033 |

Wilcoxon rank sum test of JNK activity before (averaged value from t = −6 min to t = 0
min) and after forskolin treatment (averaged value from t = 30 min to t = 36 min)

| | |
|---|---|
| P value | 0.01402 |
| Sum Rank (before forskolin treatment) | 72 |
| Sum Rank (after forskolin treatment) | 138 |
| Z value | −2.45677 |

Statistical analysis for FIG. 5B
Early M phase vs late M phase (Wilcoxon signed-rank test
with Holm-Bonferroni correction)

| | |
|---|---|
| P value | 0.0002 |
| P value summary | *** |
| Significantly different (P < 0.0167 | Yes |
| (Holm-Bonferroni corrected alpha))? | |
| One- or two-tailed P value? | Two-tailed |
| Sum of positive, negative ranks | 0.000, −91.00 |
| Sum of signed ranks (W) | −91 |
| Number of pairs | 13 |

Early G1 phase vs late G1 phase (Wilcoxon signed-rank test
with Holm-Bonferroni correction)

| | |
|---|---|
| P value | 0.000061 |
| P value summary | **** |
| Significantly different (P < 0.0125 | Yes |
| (Holm-Bonferroni corrected alpha))? | |
| One- or two-tailed P value? | Two-tailed |
| Sum of positive, negative ranks | 120.0, 0.000 |
| Sum of signed ranks (W) | 120 |
| Number of pairs | 15 |

Early S phase vs late S phase (Wilcoxon signed-rank test
with Holm-Bonferroni correction)

| | |
|---|---|
| P value | 0.0027 |
| P value summary | ** |
| Significantly different (P < 0.0167 | Yes |
| (Holm-Bonferroni corrected alpha))? | |
| One- or two-tailed P value? | Two-tailed |
| Sum of positive, negative ranks | 123.0, −13.00 |
| Sum of signed ranks (W) | 110 |
| Number of pairs | 16 |

Early G2 phase vs late G2 phase (Wilcoxon signed-rank test
with Holm-Bonferroni correction)

| | |
|---|---|
| P value | 0.0425 |
| P value summary | * |
| Significantly different (P < 0.05 | Yes |
| (Holm-Bonferroni corrected alpha))? | |
| One- or two-tailed P value? | Two-tailed |
| Sum of positive, negative ranks | 72.00, −6.000 |
| Sum of signed ranks (W) | 66 |
| Number of pairs | 12 |

Statistical analysis for FIG. 5C
Early M phase vs late M phase (Wilcoxon signed-rank test
with Holm-Bonferroni correction)

| | |
|---|---|
| P value | 0.0002 |
| P value summary | *** |
| Significantly different (P < 0.0167 | Yes |
| (Holm-Bonferroni corrected alpha))? | |
| One- or two-tailed P value? | Two-tailed |
| Sum of positive, negative ranks | 0.000, −91.00 |
| Sum of signed ranks (W) | −91 |
| Number of pairs | 13 |

Early G1 phase vs late G1 phase (Wilcoxon signed-rank test
with Holm-Bonferroni correction)

| | |
|---|---|
| P value | 0.000061 |
| P value summary | **** |
| Significantly different (P < 0.0125 | Yes |
| (Holm-Bonferroni corrected alpha))? | |
| One- or two-tailed P value? | Two-tailed |
| Sum of positive, negative ranks | 120.0, 0.000 |
| Sum of signed ranks (W) | 120 |
| Number of pairs | 15 |

Early S phase vs late S phase (Wilcoxon signed-rank test
with Holm-Bonferroni correction)

| | |
|---|---|
| P value | 0.4332 |
| P value summary | n.s. |
| Significantly different (P < 0.05 | No |
| (Holm-Bonferroni corrected alpha))? | |
| One- or two-tailed P value? | Two-tailed |
| Sum of positive, negative ranks | 52.00, −84.00 |
| Sum of signed ranks (W) | −32 |
| Number of pairs | 16 |

Early G2 phase vs late G2 phase (Wilcoxon signed-rank test
with Holm-Bonferroni correction)

| | |
|---|---|
| P value | 0.0015 |
| P value summary | ** |
| Significantly different (P < 0.025 | Yes |
| (Holm-Bonferroni corrected alpha))? | |
| One- or two-tailed P value? | Two-tailed |
| Sum of positive, negative ranks | 76.00, −2.000 |
| Sum of signed ranks (W) | 74 |
| Number of pairs | 12 |

Statistical analysis for FIG. 7B

| YFP (unpaired t test) | | Dronpa (unpaired t test) | |
|---|---|---|---|
| P value | 0.7489 | P value | 0.3484 |
| P value summary | n.s. | P value summary | n.s. |
| Significantly | No | Significantly | No |
| different | | different | |
| (P < 0.05) | | (P < 0.05) | |
| One- or two-tailed | Two-tailed | One- or two-tailed | Two-tailed |
| P value? | | P value? | |
| t, df | t = 0.3351, | t, df | t = 1.017, |
| | df = 6 | | df = 6 |
| Difference | −0.007333 ± | Difference | 0.02250 ± |
| between | 0.02188 | between | 0.02213 |
| means ± SEM | | means ± SEM | |
| 95% confidence | −0.06088 to | 95% confidence | −0.03164 to |
| interval | 0.04621 | interval | 0.07664 |
| Skylan62A (unpaired t test) | | rsFastLime (unpaired t test) | |
| P value | 0.8127 | P value | 0.2032 |
| P value summary | n.s. | P value summary | n.s. |
| Significantly | No | Significantly | No |
| different | | different | |
| (P < 0.05) | | (P < 0.05) | |
| One- or two-tailed | Two-tailed | One- or two-tailed | Two-tailed |
| P value? | | P value? | |
| t, df | t = 0.2460, | t, df | t = 1.386, |
| | df = 7 | | df = 8 |
| Difference | 0.009000 ± | Difference | −0.03800 ± |
| between | 0.03658 | between | 0.02742 |
| means ± SEM | | means ± SEM | |
| 95% confidence | −0.07751 to | 95% confidence | −0.1012 to |
| interval | 0.09551 | interval | 0.02524 |
| rsEGFP2-E (unpaired t test) | | rsGreenF-E (unpaired t test) | |
| P value | 0.7021 | P value | 0.7259 |
| P value summary | n.s. | P value summary | n.s. |
| Significantly | No | Significantly | No |
| different | | different | |
| (P < 0.05) | | (P < 0.05) | |
| One- or two-tailed | Two-tailed | One- or two-tailed | Two-tailed |
| P value? | | P value? | |
| t, df | t = 0.4111, | t, df | t = 0.3631, |
| | df = 4 | | df = 8 |
| Difference | 0.02333 ± | Difference | 0.01200 ± |
| between | 0.05676 | between | 0.03305 |
| means ± SEM | | means ± SEM | |
| 95% confidence | −0.1343 to | 95% confidence | −0.06420 to |
| interval | 0.1809 | interval | 0.08820 |

Statistical analysis for FIG. 10

| rsTagRFP (unpaired t test) | | rsFusionRed1 (unpaired t test) | |
|---|---|---|---|
| P value | 0.9261 | P value | 0.894 |
| P value summary | n.s. | P value summary | n.s. |
| Significantly | No | Significantly | No |
| different | | different | |
| (P < 0.05) | | (P < 0.05) | |
| One- or two-tailed | Two-tailed | One- or two-tailed | Two-tailed |
| P value? | | P value? | |
| t, df | t = 0.095, | T, df | t = 0.1370, |
| | df = 12 | | df = 9 |
| Difference | 0.002857 ± | Difference | 0.004000 ± |
| between | 0.03018 | between | 0.02919 |
| means ± SEM | | means ± SEM | |
| 95% confidence | −0.06290 to | 95% confidence | −0.06240 to |
| interval | 0.06862 | interval | 0.07004 |
| rScarlet (unpaired t test) | | mCherry (unpaired t test) | |
| P value | 0.1002 | P value | 0.8518 |
| P value summary | n.s. | P value summary | n.s. |
| Significantly | No | Significantly | No |
| different | | different | |
| (P < 0.05) | | (P < 0.05) | |
| One- or two-tailed | Two-tailed | One- or two-tailed | Two-tailed |
| P value? | | P value? | |
| t, df | t = 1.795, | t, df | t = 0.1917, |
| | df = 11 | | df = 10 |
| Difference | 0-0.06500 ± | Difference | 0.005000 ± |
| between | 0.03622 | between | 0.02609 |
| means ± SEM | | means ± SEM | |
| 95% confidence | −0.1447 to | 95% confidence | −0.05313 to |
| interval | 0.01472 | interval | 0.06313 |

Statistical analysis for FIG. 13
Wilcoxon rank sum test

| | |
|---|---|
| P value | 0.0015 |
| P value summary | ** |
| Significantly different (P < 0.05) | Yes |
| One- or two-tailed P value? | Two-tailed |
| Sum of ranks in data from middle G1 phase to early S phase | 145 |
| Sum of ranks in data from early S phase to late S phase | 65 |

### Molecules and Compounds

The present invention, in part, includes novel temporally multiplexed imaging (TMI) methods and components thereof, their expression in a cell, and their use for imaging in the cell, which may also be referred to herein as a “host cell.” As used herein, the term “host cell” means a cell that includes one or more components of an expressed or encoded TMI system of the invention. Non-limiting examples of components of TMI systems of the invention are described elsewhere herein. Aspects of the invention also include additional functional variants of components of TMI systems described herein, including polynucleotides, polypeptides, compositions comprising the components and functional variants thereof, and methods of using the components and functional variants thereof to perform TMI-based imaging in a cell, or in a plurality of cells. As used herein the term “plurality of cells” means more than one cell, which in some embodiments of the invention is more than 1, more than 10, more than 100, more than 1000, more than 10,000, or more than 100,000, and more than 1,000,000, including all integers within the range from 1 to at least 1,000,000.

As described herein, certain embodiments of methods and systems of the invention include expressing reporter agents with independently selected temporal properties in a cell. The term “independently selected” used herein in reference to multiple like components and characteristics in embodiments of methods and systems of the invention means selection of each like component for inclusion in the composition is selected independent of the others selected. As a non-limiting example, in a composition of the invention comprising expressing a plurality reporter agents with independently selected temporal properties, the plurality of temporal properties are considered to be “like characteristics” and each is selected independent of the others selected, meaning that the temporal properties of each the plurality of reporter agents may be selected such that each is different from all the others, selected such that all are the same, or selected such that two or more of the temporal properties may be the same as each other. As another non-limiting example, the independently selected genes included in methods and systems of the invention, are considered to be “like components” and each is selected independent of the others, meaning that the selected genes may each selected such that each is different from the others, selected such that all the genes are the same, or selected such that two or more of the genes are the same as each other. As another non-limiting example, the independently selected fluorophores included in methods and systems of the invention are considered to be “like components” and each fluorophore is selected independent of the others selected, meaning that each selected fluorophores may be different from all of the other selected fluorophores; all of the fluorophores may be the same as each other, or two or more of the selected fluorophores may be the same as each other. As another non-limiting example, independently selected reversibly photoswitchable fluorescent proteins (rsFPs) included in methods and systems of the invention are considered to be “like components” and each rsFP is selected independent of the other selected, meaning each selected rsFP may be different from all of the other selected rsFPs; all of the rsFPs may be the same as each other; or two or more of the selected rsFPs may be the same as each other. It will be understood how the term “independent selected” is applied to other components of methods and systems of the invention.

The term “associated with” used herein in reference to reporter agents and genes means the expression of the gene can be identified by the expression of the reporter agent with which it is associated. For example, in some embodiments of methods and systems of the invention, expression of the reporter agent is linked with the expression of the gene-meaning both are expressed.

Embodiments of methods and systems of the invention may also include compounds and compositions that comprise one or more components of an expressed and/or encoded TMI-based recorder system of the invention. A compound or composition that comprises a component of an expressed and/or encoded TMI system of the invention may in some embodiments, include one, two, three, four, five, six, or more additional components. Non-limiting examples of additional components are a vector, a promoter, a trafficking sequence, a delivery molecule sequence, an additional sequence, etc.

Certain embodiments of the invention include polynucleotides comprising nucleic acid sequences that encode a component of a TMI system of the invention, and some aspects of the invention comprise methods of delivering and/or using such polynucleotides in cells, tissues, and/or organisms. TMI-based component polynucleotide sequences and amino acid sequences used in aspects and methods of the invention may be “isolated” sequences. As used herein, the term “isolated” used in reference to a polynucleotide, nucleic acid sequence, polypeptide, or amino acid sequence means a polynucleotide, nucleic acid sequence, polypeptide, or amino acid sequence, respectively, that is separate from its native environment and present in sufficient quantity to permit its identification or use. Thus, a nucleic acid or amino acid sequence that makes up a component of a TMI molecular imaging molecule that is present in one or more of a vector, a cell, a tissue, an organism, etc., may be considered to be an isolated sequence if it is not naturally present in that cell, tissue, or organism, and/or did not originate in that cell, tissue, or organism.

A host cell means a cell that comprises one or more components of an expressed and/or encoded TMI molecule. In certain aspects of the invention, one or more components of an expressed and/or encoded TMI molecule of the invention are delivered into and/or expressed in a cell. Examples of cells that may be used in embodiments of the invention include, but are not limited to vertebrate cells, mammalian cells (including but not limited to non-human primate, human, dog, cat, horse, mouse, rat, etc.), insect cells (including but not limited to Drosophila, etc.), fish, worm, nematode, and avian cells. In some embodiments of the invention, a cell is a plant cell.

One or more components of a TMI system of the invention may be derived from (also referred to herein as “being a variant of”) one or more components disclosed herein, and they may exhibit the same qualitative function and/or characteristics of the TMI system component from which they have been derived, and/or may show one or more increased or decreased level of a function or characteristic of the parent component. In some embodiments of the invention an effectiveness of a variant or derived component of a TMI system set forth herein may differ from the parent component. For example, in some instances a variant or derived component is capable of increased imaging capability than is possible for its parent component.

It is understood in the art that the codon systems in different organisms can be slightly different, and that therefore where the expression of a given protein from a given organism is desired, the nucleic acid sequence can be modified for expression within that organism. Thus, in some embodiments, a polynucleotide that encodes a component of a TMI molecule or system of the invention comprises a mammalian-codon-optimized nucleic acid sequence, which may in some embodiments be a human-codon optimized nucleic acid sequence. Codon-optimized sequences can be prepared using routine methods.

### Delivery of TMI Components

Delivery of one or more components of a TMI system of the invention to a cell and/or expression of the component in a cell can be done using art-known delivery means. [See for example, Chow et al. Nature (2010); 463(7277):98-102; and for Adeno-associated virus injection: Betley, J. N. & Sternson, S. M. Hum. Gene Ther., (2011); 22, 669-677; for In utero electroporation: Saito, T. & Nakatsuji, N., Dev. Biol., (2001); 240, 237-46; for microinjection into zebrafish embryos: Rosen J. N. et al., J. Vis. Exp., (2009); (25), e1115, doi:10.3791/1115; and for DNA transfection for neuronal culture: Zeitelhofer, M. et al., Nature Protocols 2, (2007); 1692-1704, the content of each of which is incorporated by reference herein in its entirety].

In some embodiments of the invention a component of a TMI system of the invention is included as part of a fusion protein. It is well known in the art how to encode, prepare, and utilize fusion proteins that comprise a polypeptide sequence. In certain embodiments of the invention, a vector that encodes a fusion protein can be prepared and used to deliver a component of a TMI system of the invention to a cell and can also in some embodiments be used to target delivery of a component of a TMI system of the invention to a specific cell, cell type, tissue, or region in a subject. Some components of the invention include a sequence that encodes a component of a TMI system of the invention. Suitable targeting sequences useful to deliver a component of a TMI system of the invention to a cell, tissue, region of interest are known in the art. Delivery of a component of a TMI system of the invention to a cell, tissue, or region in a subject can be performed using art-known procedures. A fusion protein of the invention can be delivered to a cell by delivery of a vector encoding the TMI component. In some embodiments, the vector encodes the TMI component and in certain embodiments, the vector encodes a fusion protein comprising the TMI component. A delivered fusion protein may be expressed in a specific cell type, tissue type, organ type, and/or region in a subject, or in vitro, for example in culture, in a slice preparation, etc.

In certain aspects of the invention, a component of a TMI system of the invention is non-toxic or substantially non-toxic to the cell into which it is delivered and/or expressed. In some embodiments of the invention, a component of a TMI system of the invention is genetically introduced into a cell, and reagents and methods are provided for genetically targeted expression of components of a TMI system of the invention. Genetic targeting can be used to deliver one or more components of a TMI system of the invention to specific cell types, to specific cell subtypes, to specific spatial regions within an organism. In some embodiments of the invention, targeting can be used to control of the amount of a component of a TMI system of the invention that is expressed and the timing of the expression. Preparation, delivery, and use of a fusion protein and its encoding nucleic acid sequences are well known in the art. Routine methods can be used in conjunction with teaching herein to express one or more TMI system components and optionally additional polypeptides, in a desired cell, tissue, or region in vitro or in a subject.

### Vectors, Plasmids, and Molecules

Some embodiments of the invention include a reagent for genetically targeted expression of a component of a TMI system of the invention, wherein the reagent comprises a vector that contains the gene for the component. As used herein, the term “vector” refers to a nucleic acid molecule capable of transporting between different genetic environments another nucleic acid to which it has been operatively linked. The term “vector” may also refer to a virus or organism that is capable of transporting the nucleic acid molecule. One type of vector is an episome, i.e., a nucleic acid molecule capable of extra-chromosomal replication. Some useful vectors are those capable of autonomous replication and/or expression of nucleic acids to which they are linked. Vectors capable of directing the expression of genes to which they are operatively linked are referred to herein as “expression vectors.” Other useful vectors, include, but are not limited to viruses such as lentiviruses, retroviruses, adenoviruses, and phages. Vectors useful in some methods of the invention can genetically insert a TMI system of the invention into dividing and non-dividing cells and can insert a TMI system of the invention into an in vivo, in vitro, or ex vivo cell.

Vectors useful in methods of the invention may include additional sequences including, but not limited to one or more signal sequences and/or promoter sequences, or a combination thereof. Expression vectors and methods of their use are well known in the art. Non-limiting examples of suitable expression vectors and methods for their use are provided herein. In certain embodiments of the invention, a vector may be a lentivirus comprising the gene for a TMI system of the invention. A lentivirus is a non-limiting example of a vector that may be used to create stable cell line. The term “cell line” as used herein is an established cell culture that will continue to proliferate given the appropriate medium.

Promoters that may be used in methods and vectors of the invention include, but are not limited to, cell-specific promoters or general promoters. Methods for selecting and using cell-specific promoters and general promoters are well known in the art. A non-limiting example of a general purpose promoter that allows expression of a TMI system of the invention in a wide variety of cell types—thus a promoter for a gene that is widely expressed in a variety of cell types, for example a “housekeeping gene” can be used to express a TMI system component(s) of the invention in a variety of cell types. Non-limiting examples of general promoters are provided elsewhere herein and suitable alternative promoters are well known in the art. In certain embodiments of the invention, a promoter may be an inducible promoter, examples of which include, but are not limited to tetracycline-on or tetracycline-off, or tamoxifen-inducible Cre-E R.

In some embodiments of the invention a reagent for expression of a component of a TMI system of the invention is a vector that comprises a gene encoding the component, and optionally a gene encoding one or more additional polypeptides. Vectors useful in methods of the invention may include additional sequences including, but not limited to, one or more signal sequences and/or promoter sequences, or a combination thereof. In certain embodiments of the invention, a vector may be a lentivirus, adenovirus, adeno-associated virus, or other vector that comprises a gene encoding TMI system component(s) of the invention. An adeno-associated virus (AAV) such as AAV8, AAV1, AAV2, AAV4, AAV5, AAV9, are non-limiting examples of vectors that may be used to express a fusion protein of the invention in a cell and/or subject. Expression vectors and methods of their preparation and use are well known in the art. Non-limiting examples of suitable expression vectors and methods for their use are provided herein. Other vectors that may be used in certain embodiments of the invention are provided in the Examples section herein.

Promoters that may be used in methods and vectors of the invention include, but are not limited to, cell-specific promoters or general promoters. Non-limiting examples promoters that can be used in vectors of the invention are ubiquitous promoters, such as, but not limited to: CMV, CAG, CBA, and EF1a promoters; and tissue-specific promoters, such as but not limited to: Synapsin, CamKIIa, GFAP, RPE, ALB, TBG, MBP, MCK, TNT, and aMHC promoters. Methods to select and use ubiquitous promoters and tissue-specific promoters are well known in the art. A non-limiting example of a tissue-specific promoter that can be used to express a component of a TMI system of the invention in a cell such as a neuron is a synapsin promoter, which can be used to express the component in certain embodiments of methods of the invention. Additional tissue-specific promoters and general promoters are well known in the art and, in addition to those provided herein, may be suitable for use in compositions and methods of the invention. Other non-limiting examples of promoters that may be used in certain embodiments of methods of the invention are provided in the Examples section.

Non-limiting examples of photoswitchable fluorescent dyes that can be used in embodiments of methods and systems of the invention are provided herein. It will be understood that other photoswitchable fluorescent dyes are known in the art and routine methods can be used to include such sequences in methods and systems of the invention. Additional sequences that may be included in a fusion protein comprising a component of a TMI system of the invention are trafficking sequences, including, but not limited to: Kir2.1 sequences and functional variants thereof, KGC sequences, ER2 sequences, etc. Trafficking polypeptides and their encoding nucleic acid sequences are known in the art and routine methods can be used to include and use such sequences in fusion proteins and vectors, respectively, of the invention.

### Cells and Subjects

Some aspects of the invention include cells used in conjunction with a TMI system of the invention. Cells in which a TMI system component may be expressed, and that can be used in methods of the invention, include prokaryotic and eukaryotic cells. Certain embodiments of the invention include use of mammalian cells; including but not limited to cells of humans, non-human primates, dogs, cats, horses, rodents, etc. In some embodiments of the invention, cells that are used are non-mammalian cells; including but not limited to insect cells, avian cells, fish cells, plant cells, etc. A TMI system of the invention may be included in non-excitable cells and in excitable cells, the latter of which include cells able to produce and respond to electrical signals. Examples of excitable cell types include, but are not limited, to neurons, muscle cells, visual system cells, sensory cells, auditory cells, cardiac cells, and secretory cells (such as pancreatic cells, adrenal medulla cells, pituitary cells, etc.), cardiac cells, immune system cells, etc.

Cells in which a TMI system of the invention can be used include embryonic cells, stem cells, pluripotent cells, mature cells, geriatric cells, engineered cells, as well as cells in other developmental stages. Non-limiting examples of cells that may be used in methods of the invention include neuronal cells, nervous system cells, cardiac cells, circulatory system cells, kidney cells, liver cells, epidermal cells, visual system cells, auditory system cells, secretory cells, endocrine cells, and muscle cells.

In some embodiments, a cell used in conjunction with methods and a TMI system of the invention is a healthy normal cell that is not known or suspected of having a disease, disorder, or abnormal condition. In some embodiments of the invention, a cell used in conjunction with methods and a TMI system of the invention may in some embodiments be a normal cell or in some embodiments is an abnormal cell. Non limiting examples of elements of an abnormal cell are: (1) a cell that has a disorder, disease, or condition; (2) a cell obtained from a subject that has, had, or is suspected of having disorder, disease, or condition; (3) a cell known to be or suspected of being involved in a disorder, disease, or condition; and (4) a cell that is a model for a disorder, disease, or condition, etc. Non-limiting examples of such cells are a degenerative cell, a neurological disease-bearing cell, a cell model of a disease or condition, an injured cell, a cell downstream from a disease-bearing or injured cell, etc. In some embodiments of the invention, a cell may be a control cell. A cell that is directly or indirectly upstream from a cell in which a TMI system may be included may be a normal cell or may be an abnormal cell.
An embodiment of a TMI system of the invention may be included in a cell from or in culture, a cell in solution, a cell obtained from a subject, and/or a cell in a subject (in vivo cell). In some embodiments of the invention, a TMI system is present in and monitored in cultured cells, cultured tissues (e.g., brain slice preparations, etc.), and in living subjects, etc. As used herein, the term “subject” may refer to a human, non-human primate, cow, horse, pig, sheep, goat, dog, cat, bird, rodent, fish, insect, or other vertebrate or invertebrate organism. In certain embodiments, a subject is a mammal and in certain embodiments, a subject is a human. Additional non-limiting examples of cell types that may be used in certain methods of the invention are provided in the Examples section, as are non-limiting examples of organisms that may subjected to certain methods of the invention.

A cell that includes a TMI system and/or component of the invention may be a single cell, an isolated cell, a cell in culture, an in vitro cell, an in vivo cell, an ex vivo cell, a cell in a tissue, a cell in a subject, a cell in an organ, a cell in a cultured tissue, a cell in a neural network, a cell in a brain slice, a neuron, a cell that is one of a plurality of cells, a cell that is one in a network of two or more interconnected cells, a cell in communication with another cell, a cell that is one of two or more cells that are in physical contact with each other, etc. It will be understood that methods of the invention can be carried out in a plurality of cells such that one or more cells comprises the TMI system of the invention. Inclusion of a system of the invention in a plurality of cells permits monitoring and determining one or more alterations in expression across the plurality of cells. It will be understood that when assessing expression and history in a plurality of cells, a plurality of cells may be prepared to contain an expressed and/or encoded TMI system of the invention and the status of expression in the plurality of cells can be determined at one or more different time points by obtaining one or more cells from the plurality at the one or more different time points and determining the expression and/or history in the obtained cell or cells. At a different timepoint, another cell or other cells may be obtained from the plurality of cells and assessed. Results of two or more assessments done in cells obtained from the plurality at different times can be compared to determine a change in expression in the plurality of cells. Results using cells obtained at two or more times can be used to assess changes in expression in the plurality of cells over time and under different conditions. For example, one or more cells may be obtained from a plurality of cells comprising expressed and/or encoded TMI component or system of the invention and assessed for expression, then the plurality of cells may be contacted with a candidate stimuli and another cell or cells obtained following the contact can be assessed and compared to the initial assessment or a control assessment as a determination of the expression history of the plurality of cells.

### EXAMPLES

### Example 1

### Materials and Methods

### Molecular Cloning

Plasmids used in this study were constructed by either restriction cloning or In-Fusion assembly. Sanger sequencing was used to verify DNA sequences. The genes of Dronpa (Ando R, et al., Science, (2004); 306(5700):1370-3), YFP (Ormö M, et al., Science, (1996); 273(5280):1392-5), and mCherry (Shaner N C, et al., Nat Biotechnol., (2004); 22(12):1567-72) were amplified from addgene plasmids 57260, 1816, and 55148 respectively. The genes for rsFastLime (Stiel A C, et al., Biochem J., (2007); 402(1):35-42), rsGreenF (Roebroek T, et al., Int J Mol Sci [Internet], (2017); 18(9)), Skylan-NS (Zhang X, et al., Proc Natl Acad Sci USA, (2016); 113(37):10364-9), rsEGFP2 (Grotjohann T, et al., Elife, (2012); 1:e00248), rsTagRFP (Pletnev S, et al., J Mol Biol., (2012); 417(3):144-51), rsFusionRed1 (Pennacchietti F, et al., Nat Methods, (2018); 15(8):601-4), and GFP enhancer nanobody (Roebroek T, et al., Int J Mol Sci [Internet], (2017); 18(9)) were synthesized de novo by Integrated DNA Technologies based on the reported sequences. Site-directed mutagenesis libraries were generated using Quikchange site-directed mutagenesis (Agilent). For expression in bacteria, genes were cloned into pBAD-HisD vector. For ubiquitous expression in mammalian cells, genes were cloned into plasmids with one of the three promoters: CMV promoter, EF-1α promoter, CAG promoter. For expression in Zebrafish, genes were cloned to the pTol2-10xUAS backbone (for Gal4-dependent expression) (Köster R W, and Fraser S E, Dev Biol., (2001); 233(2):329-46).

All synthetic DNA oligonucleotides used for cloning were purchased from either Integrated DNA Technologies or Quintarabio, PCR amplification was performed using CloneAmp HiFi PCR Premix (Takara Bio). Restriction endonucleases and T4 DNA ligase were purchased from New England BioLabs and used according to the manufacturer's protocols. In-Fusion assembly master mix (Takaro Bio) was used following the manufacturer's instructions for plasmids In-Fusion assembly. Small-scale isolation of plasmid DNA was performed with plasmids mini-prep kits (Takara Bio); large-scale DNA plasmids purification was done by Quintarabio. Plasmids of rScarlet variants were isolated and purified using 96-well plasmid miniprep kits (Bioland Scientific LLC). Stellar Competent cells (Takara Bio) were used for cloning, small-scale DNA plasmids purification, and protein purification, DH5α or NEB Stable Competent cells (New England Biolabs) were used for large-scale DNA plasmids purification.

### Cell Culture and Transfection

HEK293FT cells (Thermo Fisher) were grown and maintained in Dulbecco's modified Eagle's medium (DMEM) (Gibco) supplemented with 10% heat-inactivated fetal bovine serum (Gibo), 2 mM GlutaMax (Thermo Fisher Scientific), and 1% penicillin-streptomycin (Gibco), and at 37° C. and 5% CO₂. Cells were seeded on 24-well glass-bottom plates (Cellvis) or 96-well plates (Cellvis) before transfection. Transfection of HEK293FT cells was performed when cells were 40-60% confluent with TransIT transfection reagent (Mirus Bio) according to the manufacturer's instructions. Briefly, for a 24-well plate well, 500 ng of plasmid DNA was mixed with 1.5 μl of TransIT reagent in 50 μl opti-MEM (Gibo). After 30-min incubation, the DNA and transfection reagent mix were added to the cell culture medium dropwise. Imaging was then performed 24 hours post-transfection.

U2OS cells (ATCC) were grown and maintained in McCoy's 5A medium (Gibo) supplemented with 10% heat-inactivated fetal bovine serum (Gibo) and 1% penicillin-streptomycin (Gibco), and at 37° C. and 5% CO₂. The protocols for seeding and transfection of U2OS cells were the same as those used for HEK293FT cells. For transfection of multiple constructs, plasmids were added to opti-MEM with a total amount of 500 ng and an equal ratio. The plasmid DNA was then fully mixed by vortexing before TransIT transfection reagent was added. Imaging was performed 48 hours post-transfection.

NIH/3T3 cells (ATCC) were grown and maintained in Dulbecco's modified Eagle's medium (DMEM) (Gibco) supplemented with 10% bovine calf serum (Millipore Sigma) and at 37° C. with 5% CO₂. NIH/3T3 cells were tested for mycoplasma contamination every 3 months. NIH/3T3 cells were seeded on 24-well glass-bottom plates and transfection was performed when they were 40-60% confluent using Lipofectiain 3000 (Thermo Fisher), following the manufacturer's instructions. For transfection of multiple constructs, equal amounts of plasmid constructs were fully mixed in opti-MEM (Gibo) via vortexing before the transfection reagent was added. Imaging was performed 16 to 48 hours post-transfection.

### Screening of New Reversibly Photoswitchable Fluorescent Proteins

To screen a green photoswitchable fluorescent protein with an off-switching rate between Dronpa and rsFastLime, eight variants carrying mutations at position no. 62 of Skylan were constructed and transiently expressed in HEK293FT cells individually. A 70-frame movie was then recorded for each variant using an epifluorescence inverted microscope (Eclipse Ti-E, Nikon) equipped with an Orca-Flash4.0 V2 sCMOS camera (Hamamatsu) and a SPECTRA X light engine (Lumencor). The NIS-Elements Advanced Research (Nikon) was used for automated microscope and camera control. Cells were imaged with a 40× NA 1.15 water-immersion objective lens (Nikon) at room temperature (excitation: 475/28 nm at 15 mW/mm², emission: 525/50 nm). Purple light (390/22 nm at 2 mW/mm² for 100 ms) was applied right before taking movies. The off-switching traces of each variant were extracted from 8 to 10 cells. The cells were chosen so that they were evenly distributed over the field of views. Skylan62A was the winner of the screening. The off-switching traces of rsGreenF, rsGreenF with the enhancer nanobody, rsEGFP2, and rsEGFP2 with the enhancer nanobody were also obtained using similar imaging setups and analysis (excitation: 475/28 nm at 5 mW/mm², emission: 525/50 nm).

For the screening of a new red photoswitchable fluorescent protein with a slow off-switching rate, six mutations borrowed from rsCherryRev1.4 [(Stiel A C, et al., Biophys J., (2008); 95(6):2989-97), (Lavoie-Cardinal F, et al., Chemphyschem, (2014); 15(4):655-63)] were introduced to mScarlet followed by site-directed saturation at position no. 148 and no. 162 of mScarlet (Bindels D S, et al., Nat Methods, (2017); 14(1):53-6) using the following primer:

5′atgggctggttcgcgNNCaccgagcagttgtaccccgaggacggcgtgctgaagggccttKSCaagatggccctgcgcctg-3′ (SEQ ID NO: 8). The plasmids of 196 variants were then amplified, isolated, and expressed individually in HEK293FT cells. Movies (70 frames) were then recorded (excitation: 555/28 at 9.4 mW/mm², emission: 630/75 nm; on-switching: 475/28 nm at 9.6 mW/mm² for 100 ms) for the variants with detectable fluorescence on the same wide-field microscope used for the screening of new green photoswitchable fluorescent proteins. The off-switching traces of each tested variant were then extracted from 8 to 10 cells that were evenly distributed over the field of views. The winner of the screening was named as rScarlet.

The photoswitching behaviors of commonly used red fluorescent protein mScarlet (Bindels D S, et al., Nat Methods, (2017); 14(1):53-6), mRuby2 (Lam A J, et al., Nat Methods, (2012); 9(10):1005-12), mApple (Shaner N C, et al., Nat Methods, (2008); 5(6):545-51), mCherry, mKate2 (Shcherbo D, et al., Biochem J., (2009); 418(3):567-74), tdTomato (Shaner N C, et al., Nat Biotechnol., (2004); 22(12):1567-72), TagRFP (Merzlyak E M, et al., Nat Methods, (2007); 4(7):555-7), stagRFP (Mo G C H, et al., Nat Commun., (2020); 11(1):1848), FusionRed (Shemiakina I I, et al., Nat Commun., (2012); 3:1204) and Far-red fluorescent protein mNeptune (Lin M Z, et al., Chem Biol., (2009); 16(11):1169-79), mCardinal (Chu J, et al., Nat Methods, (2014); 11(5):572-8), mMaroon (Bajar B T, et al., Nat Methods, (2016); 13(12):993-6) were measured using the same imaging setups and analysis as those for rScarlet screening. Three photoswitching cycles were measured for each FP; a pulse of blue light (475/28 nm at 9.6 mW/mm² for 50 ms) was used to switch FPs from the “off” state to the “on” state before each cycle.

### Protein Purification and In Vitro Characterization

To purify each protein sample for characterization, single E. coli colonies expressing each protein were picked and cultured in 2 mL liquid LB medium supplemented with 100 μg/mL ampicillin at 37° C. overnight. This 2-mL culture was then inoculated into a 500 ml liquid LB medium supplemented with 100 μg/mL ampicillin and 0.02% L-arabinose (wt/vol) and cultured at 28° C. for 24 h. After culture, bacteria were harvested by centrifugation. Protein purification was then performed using Capturem His-tagged purification maxiprep kit (Takara bio) following the manufacturer's instructions. Purified proteins were subjected to buffer exchange to 1× TBS (pH=7.4) with centrifugal concentrators (GE Healthcare Life Sciences).

Absorption, excitation, and emission spectra of purified Skylan62A and rScarlet were measured using Tecan Spark microplate plate. Extinction coefficients of Skylan62A and rScarlet were determined by first measuring the absorption spectrum of Skylan62A or rScarlet in 1× TBS. The concentration of each protein was then determined by measuring the absorbance of alkaline-denatured protein and assuming ε=44,000 M⁻¹ cm⁻¹ at 446 nm (Gross L A, et al., Proc Natl Acad Sci USA, (2000); 97(22):11990-5). The extinction coefficient (ε) of the protein was calculated by dividing the peak absorbance maximum by the concentration of protein. Proteins were switched to the “on” state with the illumination of purple light before each measurement.

To determine fluorescence quantum yields of Skylan62A and rScarlet, Skylan-NS and mScarlet-I were used as standard respectively. Briefly, the concentration of SKylan62A (or rScarlet) in 1× TBS was adjusted such that absorbance at the excitation wavelength was between 0.1 and 0.2. A series of dilutions of each protein solution and standard, with absorbance values ranging from 0.005 to 0.02, was prepared. The fluorescence spectrum of each dilution of each standard and protein solution was recorded and the total fluorescence intensities were obtained by integration. FPs were switched to their “on” state with the illumination of purple light before each measurement. Absorbance versus integrated fluorescence intensity was plotted for each protein and each standard. Quantum yield was calculated from the slopes (S) of each line using the equation: Φₚᵣₒₜₑᵢₙ=Φ_{standard}×(Sₚᵣₒₜₑᵢₙ/S_{standard}).

Temporal Multiplexing of rsFPs in U2OS Cells for Subcellular Labeling

Temporal multiplexing of green rsFPs: rsGreenF-E was fused with ER-targeting sequence [MLLSVPLLLGLLGLAVA (SEQ ID NO: 1)]) on the N-terminus and an ER-retention signal sequence [KDEL (SEQ ID NO: 2)] on the C-terminus (Kendall J M, et al., Biochem Biophys Res Commun., (1992); 189(2):1008-16); rsEGFP2-E was fused with histone H2B on the N-terminus; rsFastLime was fused with a mitochondria targeting sequence [MSVLTPLLLRGLTGSARRLPVPRAKIHSL (SEQ ID NO: 3)], from addgene plasmid 57287) on the N-terminus; Skylan62A was fused with Tubulin (from addgene plasmid 57302) on the C-terminus; Dronpa was fused with α-actinin (from addgene plasmid 57260) on the N-terminus, YFP was fused with LAMP1 on the N-terminus (addgene plasmid 1816) (Sherer N M, et al., Traffic, (2003); 4(11):785-801). Six more constructs were built by adding a FLAG-tag [DYKDDDK (SEQ ID NO: 4)] to the C-terminus of each FP in the previous six constructs.

The six constructs with FLAG-tag were expressed in U2OS cells individually. In parallel, one of the FLAG-tagged constructs was coexpressed with other five non-FLAG constructs in U2OS cells.

Cells with the expression of subcellular compartment-targeted FPs were then fixed with 4% PFA 48 hours after transfection followed by two washes with 1×PBS and one wash with 1×PBS containing 100 mM glycine at room temperature. Cells were permeabilized with 0.1% Triton X-100 for 10 minutes and then blocked with MAXBlock Blocking medium (Active Motif) for 15 min, followed by three washes for 5 minutes each at room temperature in 1×PBS. Next, samples were incubated with rabbit anti-FLAG antibody (Invitrogen) in MAXStain Staining medium (Active Motif) for 1 hour at room temperature followed by three washes for 5 minutes each at room temperature in 1×PBS Then, samples were incubated with Alexa647-labeled goat-anti-rabbit antibody (Abcam) in MAXStain Staining medium (Active Motif) for 1 hour at room temperature followed by three washes for 5 minutes each at room temperature in 1×PBS. Samples were then stored in 1×PBS and imaged on a Nikon Eclipse Ti inverted microscope equipped with a confocal spinning disk (CSU-W1), a 40×, 1.15 NA water-immersion objective, and a 5.5 Zyla camera (Andor), controlled by NIS-Elements AR software.

For the imaging of the cells with only one FLAG-tagged construct expressed, two snapshot images were taken from green channel (exposure time 50 ms, excitation: 488 nm, emission: 525/30 nm) and far-red channel (exposure time 50 ms, excitation: 637 nm, em: 700/50 nm) for each FOV. A colocalization test was then run between the two images from the same FOV to get a Pearson's correlation value. For the imaging of the cells with six constructs expressed (one FLAG-tagged construct plus five constructs without FLAG tag), a 70-frame movie and a snapshot image were taken from green (exposure time 50 ms, excitation: 488 nm at 40 mW/mm², emission: 525/30 nm) and far-red channel, respectively for each FOV. Six unmixed images were obtained via signal unmixing of each movie. The unmixed image from the FLAG-tagged construct was then colocalized with the image taken from the same FOV via far-red channel to get a Pearson's correlation value. Purple light (405 nm at 9.7 mW/mm²) was applied for 50 ms before the movies were taken.

Temporal multiplexing of red FPs: rsTagRFP was fused with histone H2B (same as the tag fused to rsEGFP2-E in the previous experiment) on the N-terminus, rsFusinoRed was fused to Human Vimentin Sequence (from addgene plasmid 57306) on the C-terminus. rScarlet was fused with α-actinin (same as the tag fused to Dronpa in the previous experiment) on the N-terminus. mCherry was fused with a mitochondrial targeting sequence (same as the tag fused to rsFastLime in the previous experiment) on the N-terminus. Four more constructs were built by adding a FLAG-tag [DYKDDDK (SEQ ID NO: 4)] to the C-terminus of each FP in the aforementioned four constructs.

The protocols for cell transfection, fixation, and immunostaining were the same as those for green FPs. Imaging of red FPs was also performed on the same microscope as the imaging of the green FPs. For the imaging of the cells with only one FLAG-tagged construct expressed, two snapshot images were taken from red channel (exposure time 100 ms, excitation: 561 nm, emission: 579/34 nm) and far-red channel (exposure time 50 ms, excitation: 637 nm, emission: 700/50 nm) for each FOV. A colocalization test was then run between the two images from the same FOV to get a Pearson's correlation value. For the imaging of the cells with four constructs expressed (one FLAG-tagged construct plus three constructs without FLAG tag), a 70-frame movie and a snapshot image were taken from red (ex: 561 nm at 50 mW/mm², emission: 579/34 nm) and far-red channel, respectively for each FOV. Four unmixed images were obtained via signal unmixing of each movie. The unmixed image from the FLAG-tagged construct was then co-localized with the image taken from the same FOV via far-red channel to get a Pearson's correlation value. Cyan light (488 nm at 40 mW/mm²) was applied or 50 ms before movies were taken.

Crosstalk measurements: For the crosstalk measurements of green FPs, seeded NIH3T3 cells were transfected with pcDuex2-rsGreenF-E, pcDuex2-rsEGFP2-E, pcDuex2-rsFastLime, pcDuex-Skylan62A, pcDuex2-Dronpa, and pcDuex2-YFP separately in six different wells of 24-well plates. Three hours after transfection, the growth medium with transfection reagents of each well was aspirated and the cells were then washed with 1× PBS three times followed by trypsin treatment (0.05% trypsin-EDTA(Gibo)) for 2 mins. The detached cells from each well were then suspended and collected before being fully mixed with the cells from the other five wells. Mixed cells were then seeded back to 24-well plates. 16-24 hours after re-seeding, imaging was performed on a Nikon Eclipse Ti inverted microscope equipped with a confocal spinning disk (CSU-W1), a 20×, 0.75 NA air objective, and a 5.5 Zyla camera (Andor), controlled by NIS-Elements AR software. For each FOV, a 70-frame movie was taken (exposure time, 50 ms, excitation: 488 nm at 10 mW/mm², emission: 525/30 nm, on-switch: 405 nm at 2.5 mW/mm² for 100 ms) and six unmixed images were obtained via signal unmixing. Since each transfected cell only expressed one FP, the crosstalks of the expressed FP to other FPs were calculated as the percentages of the fluorescence of other FP channels in that cell to the fluorescence of the expressed FP channel in the same cell.

The crosstalk measurements of red FPs were similar to that of green FPs except that only four plasmids (pcDuex2-rsTagRFP, pcDuex2-rsFusionRed1, pcDuex2-rSarlet, pcDuex2-mCherry) were used and the movies were taken using red channel (exposure time: 100 ms, excitation: 561 nm at 12.5 mW/mm², emission: 579/34 nm, on-switch: 488 nm at 10 mW/mm² for 100 ms).

### Temporal Multiplexing Simulation

For simulations of temporal multiplexing, we used a pre-acquired fluorescent image (the image was taken on a Nikon epifluorescence inverted microscope with a 20×, 0.75 NA air objective; image size: 1024×1024) of NIH/3T3 cells expressing Dronpa to generate movies for both green FPs and red FPs. Segmentation was first applied to the fluorescent image to convert Dronpa-expressing cells to cell-shaped masks. Then, the normalized traces of six green FPs (or four red FPs) were scaled, each with a random ratio (the sum of the ratios equals 1) to create a hybrid trace for each mask (different masks contain different ratios of the six FPs, same masks contain the same ratios of the six FPs). Next, the values of hybrid traces (ranging from 0 to 1) at each time point (70 time-points in total) were used to multiply the fluorescence value of each pixel within the cell-shaped masks to generate a 70-frame movie (the fluorescence of the non-masking area was assigned as 0). Poisson noises were then calculated according to the fluorescence intensity at each pixel and then applied back to each pixel of the movie. In the meantime, six ground truth images (or four ground truth images for red FPs) were generated by multiplying the randomly assigned ratio of each FP by the fluorescence of the pre-acquired fluorescence image at each pixel within the cell-shaped masks. The fluorescence of the FPs in the non-masking area was assigned as 0.

### Zebrafish Imaging

All experiments were conducted in accordance with MIT Committee on Animal Care. Zebrafish were raised and bred at 28° C. according to standard methods. DNA plasmids encoding rsTagRFP, rsFusionRed1, and rScarlet under the control of the 10×UAS promoter were mixed with a ratio of 2:2:1 and co-injected with Tol2 transposase mRNA into embryos of the pan-neuronal expressing Gal4 line, Tg(elavl3:GAL4-VP16) (Kimura Y, et al., Development, (2008); 135(18):3001-5). Briefly, DNA and Tol2 transposase mRNA, synthesized using pCR2FA as a template (Kwan K M, et al., Dev Dyn., (2007); 236(11):3088-99) (mMESSAGE mMACHINE SP6 Transcription Kit, Thermo Fisher), was diluted to a final concentration of 25 ng/μl in 0.4 mM KCl solution containing 0.05% phenol red solution (Millipore Sigma) to monitor the injection quality. The mixture was kept on ice to minimize the degradation of mRNA during the injection. The mixture was injected into embryos at 1-4 cell stages (Fisher S, et al., Nat Protoc., (2006); 1(3):1297-305). Larvae were screened for red fluorescence in the brain and spinal cord at 2-3 days post-fertilization (animals were used without regard to sex) and subsequently imaged on day 5 after fertilization. To image zebrafish larvae, larvae were immobilized in 1.5% ultra-low-melting agarose (Millipore Sigma) prepared in E3 medium and paralyzed with 0.2 mg/ml pancuronium bromide (Millipore Sigma). Imaging was performed on a Nikon Eclipse Ti inverted microscope equipped with a confocal spinning disk (CSU-W1), a 40×, 1.15 NA water-immersion objective, and a 5.5 Zyla camera (Andor), controlled by NIS-Elements AR software. A 60-frame movie was taken for each FOV (exposure time 100 ms, excitation: 561 nm at 50 mW/mm², emission: 579/34 nm, on-switch: 488 nm at 40 mW/mm² for 50 ms).

### Imaging of Cell Cycle Phases and Kinase Activities in NIH3T3 Cells

FUCCI4 constructs were gifts from Michael Z Lin (addgene no. 83841-83942) (Bajar B T, et al., Nat Methods, (2016); 13(12):993-6). Single-color FUCCI4 was built by replacing Clover, mKO2, mMaroon1, and mTurquoise2 with rsGreenFast-E, Dronpa, Skylan62A, and YFP respectively. 16-24 hours after transfection, NIH/3T3 cells were imaged on an epifluorescence inverted microscope (Eclipse Ti-E, Nikon) equipped with a 20×, 0.75 NA air objective, Perfect Focus System, an Orca-Flash4.0 V2 sCMOS camera (Hamamatsu), and a SPECTRA X light engine (Lumencor). Cells were placed in a stage-top incubator with a controlled environment at 37° C. and 5% humidified CO₂ (Live Cell Instrument), and movies (60 frames in 15 s, excitation light was on during the whole 15s) were acquired every 30 min.

Excitation: 475/28 nm at 9.6 mW/mm², emission: 525/50 nm, exposure time: 50 ms, on switching: 390/22 nm at 1.2 mW/mm² for 100 ms.

The constructs of JNKKTRrsGreenFast-E (JNKKTR is SEQ ID NO: 5), P38KTRDronpa, ERKKTRrsFastLime (ERKKTR is SEQ ID NO: 6) were built based on the original KTRs constructs JNKKTRClover (addgene plasmid 59151; JNKKTR is SEQ ID NO: 5), P38KTRmCerulean3 (addgene plasmid 59155), ERKKTRClover(addgene plasmid 59150; ERKKTR is SEQ ID NO: 6), all gift of Markus W. Covert (Regot S, et al., Cell, (2014); 157(7):1724-34). The newly developed three KTRs were then used along with PKAKTRClover (addgene plasmid 59151; PKAKTR is SEQ ID NO: 7) and H2B-TagBFP to report the activities of all four kinases. H2B-TagBFP was used as a nucleus marker. NIH/3T3 cells were imaged on the same microscope and incubator system as the imaging of cell cycle phases. Cell culture media were changed to imaging media (MEM (Gibo) without phenol red with 1% FBS (Gibo)) prior to imaging. The imaging conditions (including exposure time, excitation, and emission) for KTRs were the same as those for imaging cell cycle phases as described previously. Images of H2B-TagBFP (excitation: 390/22 nm at 1.2 mW/mm²; emission: 447/60 nm, exposure time: 100 ms) were acquired right before recording movies to switch rsFPs to the “on”-state. Images and movies were acquired every 2 min. 50 μM Forskolin (Millipore Sigma), and 20 ng/ml basic fibroblast growth factor2 (bFGF2, R&D System) were used to activate kinase activities.

### Combined Temporal Multiplexing and Spectral Multiplexing

Simultaneous imaging of cell cycle phases and activity of cyclin-dependent kinases: To increase the number of genes co-expressed within single cells, the four genes encoding FUUCI4 were cloned into a single plasmid as the following: CMV-rsGreenFast-Geminin₁₋₁₁₀-P2A-Dronpa-cdt₃₀₋₁₂₀-IRES-H1-Skylan62A-P2A-YFP-SLBP₁₈₋₁₂₆. NIH3T3 cells were then transfected with the aforementioned plasmid, plasmid EF1α-DHB-TagBFP2 (Spencer S L, et al., Cell, (2013); 155(2):369-83), and plasmid EF1α-mCherry-CDK4KTR (Yang H W, et al., Elife [Internet], (2020); 9). Imaging was performed 16-24 hours post-transfection on the same epifluorescence inverted microscope and incubation system as described previously. Blue channel (excitation: 390/22 nm at 1.2 mW/mm²; emission: 447/60 nm, exposure time: 100 ms), green channel (60 frames in 15 s, excitation light (475/28 nm at 9.6 mW/mm²) was on during the whole 15s, emission: 525/50 nm; explode time: 50 ms) and red channel (excitation: 555/28 at 9.4 mW/mm², emission: 630/75 nm; exposure time: 100 ms) were used together for imaging of 6 signals. Purple light illumination used in the blue channel for excitation also served as the “on” trigger for green rsFPs. Images and movies were acquired every 30 min for 24-48 hours without stimulation.

Simultaneous imaging of seven cell activities within single cells: The genes of the single-color KTRs were cloned into the following plasmid: CAG-JNKKTRrsGreenF-E-P2A-ERK KTRrsFastLime-IRES-PKAKTRClover-P2A-P38KTRDropna (JNKKTR is SEQ ID NO: 5 and PKAKTR is SEQ ID NO: 7). The aforementioned plasmid was then used with plasmid CAG-NIR-GECO2G (Qian Y, et al., PLoS Biol., (2020); 18(11):e3000965), plasmid CMV-Pink Flamindo (Harada K, et al., Sci Rep., (2017); 7(1):7351), and plasmid CMV-BlueCKAR (Mehta S, et al., Nat Cell Biol, (2018); 20(10):1215-25) for imaging 7 signals within individual NIH/3T3 cells. Imaging conditions for blue channel, green channel, and red channel were the same as those used in the imaging of cell cycle phases and CDKs activities. An extra channel (excitation: 637/12 nm at 9 mW/mm²; emission: 664LP, exposure time: 100 ms) was used for imaging NIR-GECO2G. Images and movies were acquired every 2 min. 50 μM forskolin (Millipore Sigma) and 100 ng/ml Phorbol 12-myristate 13-acetate (PMA) (Millipore Sigma) were used to stimulate cells.

### Signal Unmixing and Image Analysis

Signal unmixing of temporal multiplexed imaging: Reference traces of FPs used for signal unmixing were collected right before or after each imaging experiment. Each reference trace was an averaged result from 10 to 30 cells of 2 to 3 movies from one cell culture batch. Trace from each cell was normalized to the maximum value before averaging. Cells were selected so they were evenly distributed on the field-of-views. For signal unmixing of temporal multiplexing imaging, the recorded trace at each pixel was first normalized to the maximum value and then unmixed into a linear combination of the reference traces of fluorophores using least squares regression. Next, the resultant ratios (ranging from 0 to 1) of fluorophores at each pixel are multiplied by the fluorescence value of the first frame of the movie at this very pixel to generate unmixed images. The code for signal unmixing is available at github.com/qiany09/Temporally-Multiplexed-Imaging.

Movies were processed in Fiji as follows before being subjected to signal unmixing using custom Matlab code (or before being used for extracting reference traces): images were down-sampled from size 2048×2048 to size 1024×1024 or size 512×512 (to decrease computing time) followed by background subtraction.

Quantification of KTRs: Kinase activities reported by KTRs including CDK2 reporter and CDK4/6 reporter were quantified following the methods described previously (Regot S, et al., Cell, (2014); 157(7):1724-34). Briefly, to calculate cytoplasmic intensity to nuclear intensity, a nucleus and a five-pixel-wide cytoplasm ring were segmented for each cell via nucleus-targeted fluorescent proteins. Nucleus segmentation and cell tracking were performed in Fiji using StarDist (Schmidt U, et al., Springer International Publishing, (2018); 265-73) and trackmate, respectively. Cytoplasmic rings were segmented by using a custom code in Fiji. Median intensity extracted from each ROI was used to calculate ratios. The ratios reflect kinase activity. CDK4/6 activity was then corrected by deducting 0.35-fold CDK2 activity (Yang H W, et al., Elife [Internet], (2020); 9).

Analysis of CDK2 and CDK4/6 activity traces (FIG. 5B, C): CDK traces of each cell were first divided into sub-traces of different cell cycle phases. Each sub-trace of a complete cell cycle phase was then evenly split into three parts. A value was obtained by averaging all the data points in one part, thus 3 values representing CDK activity at early, middle, and late stage of a cell cycle phase were obtained for a given cell cycle phase of a given cell.

All images in the manuscript were processed and analyzed using Fiji. Traces and graphs were generated using GraphPad prism 8 or Origin9.0.

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention. All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements, and/or steps are in any way required for one or more embodiments or that one or more embodiments necessarily include logic for deciding, with or without other input or prompting, whether these features, elements, and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

All references, patents and patent applications and publications that are cited or referred to in this application are incorporated by reference in their entirety herein.

1. A method of measuring one or more cell activities, the method comprising
(a) expressing in a cell a plurality of reporter agents with independently selected temporal properties, wherein each of the expressed reporter agents is associated an independently selected gene;
(b) obtaining simultaneous images of the plurality of expressed reporter agents;
(c) linearly unmixing the obtained simultaneous images; and
(d) analyzing the unmixed images,
wherein the analysis comprises measuring the one or more cell activities.

2. The method of claim 1, wherein the reporter agents each comprises an independently selected fluorophore.

3. The method of claim 1, wherein the expressed reporter agent indicates expression of its associated independently selected gene.

4. The method of claim 1, wherein one or more of the plurality of the reporter agents comprises an independently selected reversibly photoswitchable fluorescent protein (rsFP).

5. The method of claim 4, wherein the reversibly photoswitchable fluorescent proteins (rsFPs) possess different off rates and indicate the expression of their independently selected genes.

6. The method of claim 1, wherein the linearly unmixing of the images separates the plurality of reporter agent signals from each other.

7. (canceled)

8. The method of claim 1, wherein the cell activity comprises one or more of an enzyme activity in the cell, a cell cycle signal activity in the cell, or a second messenger activity in the cell.

9. The method of claim 1, further comprising determining a relationship between two or more of the measured cell activities, optionally wherein the determined relationship comprises a relationship among two or more second messengers, kinases, and cell cycle signals in the cell.

10. (canceled)

11. The method of claim 1, wherein the cell is a vertebrate cell.

12. (canceled)

13. A temporally multiplexed imaging (TMI) system, the system comprising an encoded plurality of reporter agents with independently selected temporal properties expressed in a cell, wherein each of the expressed reporter agents is associated with an independently selected gene, and each expressed reporter agent indicates expression of its associated independently selected gene.

14. The system of claim 13, wherein the reporter agents comprise independently selected fluorophores.

15. The system of claim 13, wherein one or more of the plurality of the reporter agents each comprises an independently selected reversibly photoswitchable fluorescent protein (rsFP).

16. The system of claim 15, wherein the reversibly photoswitchable fluorescent proteins (rsFPs) possess different off rates and indicate the expression of their independently selected genes.

17. The system of claim 13, wherein the reporter agents are expressed in a cell and images of the reporter agents are obtained.

18. The system of claim 17, wherein the obtained images are linearly unmixed.

19. The system of claim 18, wherein the linearly unmixing separates the plurality of reporter agent signals from each other.

20. (canceled)

21. The system of claim 18, wherein the linearly unmixed images are analyzed.

22. The system of claim 21, wherein the analysis comprises measurement of one or more of a cell activity in the cell, optionally wherein the cell activity comprises one or more of an enzyme activity in the cell, a cell cycle signal activity in the cell, or a second messenger activity in the cell.

23. (canceled)

24. The system of claim 22, wherein the measurement of two or more of the cell activities determines a relationship between the two or more of activities in the cell, optionally wherein the determined relationship comprises a relationship among two or more second messengers, kinases, and cell cycle signals in the cell.

25. (canceled)

26. The system of claim 13, wherein the cell a vertebrate cell.

27. (canceled)


SEQUENCE LISTING (7 sequences)

SEQ ID NO: 1 - protein, 17 aa, synthetic construct
MLLSVPLLLGLLGLAVA

SEQ ID NO: 2 - protein, 4 aa, synthetic construct
KDEL

SEQ ID NO: 3 - protein, 29 aa, synthetic construct
MSVLTPLLLRGLTGSARRLPVPRAKIHSL

SEQ ID NO: 4 - protein, 7 aa, synthetic construct
DYKDDDK

SEQ ID NO: 5 - protein, 6 aa, synthetic construct
JNKKTR

SEQ ID NO: 6 - protein, 6 aa, synthetic construct
ERKKTR

SEQ ID NO: 7 - protein, 6 aa, synthetic construct
PKAKTR